(19) United States
(12) Patent Application Publication
(10) Pub. No.: US 2015/0359857 A1
(43) Pub. Date: Dec. 17, 2015

(54) Aerosolized fibrin hemostat

(71) Applicants: George David Falus, New York City, NY (US); Leonid Medved, Ellicot City, MD (US)

(72) Inventors: George David Falus, New York City, NY (US); Leonid Medved, Ellicot City, MD (US)

(73) Assignee: Biomedica Management Corporation, Halethorpe, MD (US)

(21) Appl. No.: 14/247,005

(22) Filed: Apr. 7, 2014

(51) Int. Cl. A61K 38/45; A61M 11/02; A61K 38/36

(52) U.S. Cl. CPC A61K 38/45; A61K 38/36; A61M 11/02; C12Y 203/02013; A61M 2202/064; A61M 2202/0468

(57) ABSTRACT

ClotSpray is a sprayable fibrin-based hemostatic agent applied from an aerosol can. The agent is intended for use as an adjunct or primary treatment in moderate intraoperative hemorrhage and treatment of burns. It can be applied topically to the wound from an aerosol can without need of preparation, unfreezing or interruption during the length of the surgical procedure. Its crosslinking technology generates an adhesive fibrin sealant required for hemostasis. The attachment properties of the gel as well as the instant formation of a fibrin gel ensures that a strong stable fibrin clot will control light to moderate bleeding from a wound.

### RELATED APPLICATIONS

This application is a Continuation-In-Part of U.S. patent application Ser. No. 13/731,126 filed on Dec. 31, 2012 which is a continuation in part of U.S. Pat. No. 8,367,802. All description, drawings and teachings set forth therein are expressly incorporated by reference herein and we claim priority upon the teachings expressly made therein.

### FIELD OF THE INVENTION

The present invention is generally related to an adhesive sealant and hemostatic agent presented in the form of a two-solution composition that can be sprayed from an aerosol-like device. The invention is particularly related to a fibrin monomer in acid solution produced by a dialysis method in industrial quantities, which is polymerized by a transglutaminase containing basic buffer applied over the wound to seal tissue, in order to prevent or control intracavitary, vascular, epidermal or internal hemorrhage. The spray could be used also as carrier of therapeutic agents (e.g. antibiotics)

### BACKGROUND OF THE INVENTION

The present invention is intended to be used in cases of moderate bleeding following burn decortication, organ resection, vascular repair, soft tissue bleeding or dermal wounds that often require an adjunct to control the bleeding.

### Current Solutions and Limitations.

As a result of their hemostatic and adhesive properties, fibrin sealants, or thrombin-based products have been extensively used in surgical specialties for over two decades in order to reduce blood loss and post-operative bleeding. Several technologies and products have been developed for the formulation of hemostats; some of them contain biological materials such as collagen or fibrinogen and thrombin, others are made of synthetic materials.

Plasma derived products using thrombin or fibrin are biological adhesives that have the ability to mimic some of the processes observed in the natural coagulation cascade (4). Fibrin products are characterized for their ability to adhere to human tissue as they polymerize to form a blood clot (1, 2, 3), although the physicochemical characteristics of the fibrin product may vary widely depending on the manufacturing process and components included in the formulation. These compounds are used to seal wounds that have been sutured or stapled, or diffuse bleeding from surgical or traumatic laceration, decortication or debris removal such as in the case of severe burns. They are generally compressed against an injured area until a stable clot is formed. The strength of the clot, the shelf life, the storage conditions as well as other biochemical and mechanical properties have a critical effect upon the efficacy, safety and ease of use of the fibrin-based sealant. The main components of fibrin sealants are fibrinogen, thrombin, calcium chloride, plasmatic proteins, factor XIII and stabilizers that prevent premature polymerization and degradation. The components are often extracted from human plasma or produced by recombinant techniques. Mixing fibrinogen and thrombin creates a polymer barrier (fibrin) that simulates the last stages of the natural coagulation cascade, which is the formation of a structured fibrin clot that encloses platelets and blood cells. Biodegradable supports are often added to these products when compression is needed.

There are several commercial products available (Floseal, Gelfoam, Evicel) (3, 5). However, these products have limitations in terms of adverse events resulting from the use of certain components (e.g. inflammatory effects from preservatives and immune reactions from thrombin), impractical storage conditions (freezing and thawing), time consuming steps for preparation before usage, and costs resulting from formulations with high concentrations of fibrin in order to speed up the polymerization time. One of the major limitations encountered in the development and/or use of fibrin compositions is their inability to form a sufficiently strong bond to tissues in the midst of flowing blood, and to instantly produce a clot following application when the fibrin component concentration is below 80 mg/ml. To improve the hemostatic efficacy, manual tamponade or compression is usually required which is not practical in many surgical situations (i.e. laparoscopic surgery) and particularly when aerosol spraying is used as a method of application. Indeed, current fibrin based products are not effective unless produced with a formulation of high fibrinogen (over 80 mg/mL) and thrombin concentration (over 500 units per ml), which substantially increases the manufacturing cost, delays degradation, and can result in adverse events. Also, there are many situations where the use of compression, sutures and/or staples is undesirable, inappropriate or impossible, (e.g. in extensive bone, brain, vascular, interventional radiology, retroperitoneal and spine surgery and the treatment of burns), as well as the need to cover extensive areas of wound which would be impractical with current fibrin sealants. In addition, the inclusion of thrombin prevents the recovery of lost blood at the risk of creating coagulation of the blood, which can clog and damage the recovering equipment and filters.

The present alternative approach: The method to produce an aerosolized fibrin sealant described in this patent is based in the composition, manufacturing process, and a method of application of a fibrin monomer in acid solution that can be instantly polymerized by a neutralization buffer when it is sprayed by an aerosol-type device. In order to form a strong physical barrier that resists the flow of blood, the monomer must polymerize as soon as it is released by the aerosol valve, to produce a fibrin clot that is bonded to tissues in the midst of flowing blood, and remain at the lacerated site. The ability of this fibrin monomer-based sealant to adhere to human tissue is critically dependent of the composition of solutions, method of production of fibrin monomer solution, method of neutralization, and method of application, all of which results in the rapid formation (less than 3 seconds) of a fibrin polymer with mechanical properties mainly defined, but not exclusively, by a G′ rheometry value higher than 15,000 dyn/cm2, which can withstand the forces exerted by flowing blood and by embedded cells. Unlike most sealants currently produced, the present invention does not resort to the mixture of fibrinogen and thrombin in situ, but to the polymerization of a fibrin monomer solution by a change of pH from acid to neutral. (6) Although both neutralization of fibrin monomer in acid solution and cleavage of fibrinogen by thrombin lead to the formation of a fibrin polymer, the physicochemical and mechanical characteristic of the resulting fibrin vary to the extent that some forms of fibrous gels cannot practically perform as hemostats. The properties of clots vary greatly depending on the conditions of polymerization (Weisel 2007a). The thickness of fibers, the extent of branching and the pore size of clots can vary to a very large, extent, depending on a whole host of factors. For example, the thrombin concentration can have dramatic effects on dot structure, with low thrombin yielding dots with thick fibers, few branch points and large pores. Similarly, salt concentration, pH and many plasma proteins affect clot structure and properties (5). Many studies have shown that clots of pure fibrin are extensible and elastic (9, 10).

All these factors determine the ability of a hemostat to maintain hemostasis under blood flowing conditions; the fibrin polymer must be formed within 3 seconds, have suitable stiffness and plasticity (4), but also sufficient permeability so that the network can be effectively decomposed (lysed) by proteolytic enzymes (5, 6). It is generally accepted that the properties of these fibers underlie the mechanical properties of clots in vivo (2). For example, studies with mice lacking fibrinogen have shown that clots formed in the absence of a fibrin network failed to resist shear stress (3). Such clots grow to a certain size at the site of injury, then break off and flow downstream. Because the mechanical performance of the fibrin gel is critical to its physiological function, the method to produce the fibrin monomer in solution as well as the composition of the solution characterizes the fibrin monomer beyond the common designation of desAB fibrin or fibrin II. The fibrin monomer solution described in this patent will be called thereof “ACstrength Fibrin II monomer solution” defined by a specific and superior polymerization rate and by mechanical properties of the fibrin formed by the polymerization of AC Fibrin II monomer solution. As indicated in EXAMPLE 1 the viscoelastic properties of the various monomer solutions (e.g. acetic acid,glycine buffer) produce widely different fibrin meshes with varying strengths. Viscoelastic materials such as a fibrin clot are well characterized by the way these materials store deformation energy, as quantified by the elastic modulus G′, but also by how they dissipate energy, as quantified by the viscous modulus G″. Fibrin gels are among the most resilient polymer gels in nature (4,5). They stiffen strongly when deformed and thereby become increasingly resistant to further deformation (12).

The mechanical properties of the fibrin at a concentration of 25 mg/ml to 60 mg/ml formed by the polymerization of ACstrength Fibrin II monomer solution are defined by the dynamic shear storage moduli G′ over 15,000 dyn/cm² measured with a strain-controlled rheometer at a maximal strain amplitudes showing the strain stiffening of the fibrin gel under shear stress (EXAMPLES 2a and 2b).

The ACstrength Fibrin II monomer solution is also characterized by a monomeric condition indicating that a minimal amount of protofibrils or aggregates exist in solution, (EXAMPLE 3 and EXAMPLE 8) clottability over 99.7%, and clotting time below 12 sec. at a concentration of 12 mg/ml, and between 3 and 5 seconds at a concentration of 40 mg/ml (EXAMPLE 4). At similar concentrations the neutralization of AC strength fibrin monomer II solution produced under the methods described in U.S. Pat. No. 8,367,802 (G Falus et al.) and thereof improved, and neutralized by the neutralization buffer described in the present patent occurs 5 times faster that the mix of thrombin with fibrinogen and 100% stronger as defined by G′.

A narrow range of critical pH between 3.4 and 3.8 determines the optimal clotting time for an effective application over flowing blood. Indeed fibrin monomer can only preserve its native structure in a narrow pH range, namely, at pH 3.0-4.2. Optimal clotting properties for a polymerization time in the range of 6 to 8 seconds at concentrations of 40 mg/ml occur at a pH range of 3.4 to 3.8 (EXAMPLE 5).

The ACstrength Fibrin II monomer solution is with is stable for 150 days with no preservatives or additives when stored at 4° C., where stability is defined as the achievement of below 10 sec in clotting ting time, 99.3% in Clottability, and showing formation of d-dimmers and oligomers by SDS-PAGE. The fibrin monomerization allows for long-term storage, transportation and readiness.

Unlike fibrinogen at a concentration of 80 mg/ml, the two solutions used in the composition—monomer and neutralization buffer—are stable in refrigerated conditions with a viscosity close to water, allows for application through a simple spray device similar to the ones used in the household. They can be sterilized by simple filtration, and the polymer formed is defined as biocompatible or not interfering with cell morphology, biodegradable in that is degraded by body enzymes, and non-toxic for human use. When mixed and sprayed through an aerosol-like device, the solutions create in-situ a three-dimensional crosslinked polymer that unlike fibrins crosslinked exclusively by Factor XIII, is non-specific, crosslinking two of the three chains, and forming a fibrin network that is bonded to the tissue as a strong fibrin clot, partly stabilized by transglutaminase enzyme present in the buffer, or alternatively stored in solid form in the mixing path, and further stabilized by Factor XIII present in the blood (EXAMPLE 10).

Aerosol Device. The aerosol device described in this invention offers important advantages such as easy of use and storage over the historically used dual-syringe method of application of fibrin sealants while addressing safety concerns. Because solutions in contact with gas inside an aerosol represent a significant risk of embolism in the application of fibrin glues as well as to the integrity of the components, a novel method that avoids contact with the pressurizing gas has been developed. The solutions a) fibrin monomer and b) neutralization buffer are stored in Sterifill syringes manufactured by Beckton Dikinson or in other syringes produced with very low leachable plastics, inside the aerosol.

The invention provides a safe biomaterial applicator that uses aerosol cans, and aerosol valve technology to displace a piston within a syringe containing the biomaterial component. The displacement of the piston within the syringe can occur with a high degree of force, and at a displacement rate dependent on the pressurization, as to eject the biomaterial(s) into a common manifold that will mix and spray it into a surgical site.

The invention can optionally attach a small container to the mixing tip or other sections of the fluid path in order to store within the flow path solid materials such as tranglutaminase enzyme, antibiotics or other therapeutic agent that could be dissolved by the biomaterial and incorporated in the spray. In addition, the invention supports the ability to connect multiple individual aerosol cans, containing different components of a biomaterial with their respective individual syringes.

Composition. The present technology comprises fibrin monomer in acetic acid solution at a concentration of 20 mg/ml to 60 mg/ml ready to polymerize at change of pH when mixed with neutralization buffer in the volume proportion of 1:1; or 1:3. The concentration value is critical since at lower concentration the fibrin polymer is formed too slowly for therapeutic use, and at a higher concentration it aggregates in solution. The ACstrength Fibrin II monomer solution is produced by the dialysis method described in U.S. Pat. No. 8,367,802 G Falus et al.), or alternatively by the dynamic dialysis method below described.

The neutralization buffer solution is composed of HEPES, NaCl, and CaCl₂. The neutralization buffer changes the monomer pH from 3.5 to 6.8-7.2 inducing the fibrin polymerization. Both solutions delivered through an aerosol-like device below described allows for the almost instant polymerization of the mix. It polymerizes almost instantly creating a mesh of fibrin fibers that form the fibrin clot at site of injury (7). (EXAMPLE 4) The addition of calcium independent tranglutaminase enzyme (ACTIVA) strengthens the covalent bonds of the polymer formed over the wound. Under coagulant conditions activated Factor XIII, present in the blood, also contributes to this process by stabilizing the fibrin clot through fastening the crosslinking of the gamma chain. Alternatively, the ACstrength Fibrin II monomer solution can be neutralized by mixing through a double-barrel syringe when instant polymerization is not required for therapeutic use (e.g. fibrin glue ptyergium surgery)

### Optimization Process for the Production of Fibrin Monomer Solution

An experimental method for producing fibrin monomer was first described and published by Belitser et al (1968, BBA) (11). Such method limits the production of monomer to a few milligrams per day. Although U.S. Pat. No. 5,750,657 Edwardson et al. describes a method of preparing a fibrin sealant utilizing a fibrin monomer composition, the present invention resorts to a fibrin polymer obtained by the polymerization of fibrin monomer solution produced by the dialysis method (U.S. Pat. No. 8,367,802 Falus et al), and herewith improved to obtain a monomer solution in industrial quantities at concentrations of 25 mg/ml to 60 mg/ml in abbreviated processing time, that polymerizes very rapidly with a specific gel strength and binding properties. U.S. Pat. No. 8,367,802 Falus et al describes method to produce fibrin monomer in acetic acid solution at a concentration of 5 mg/ml by dissolving fibrin II polymer through static dialysis, and thereof increasing the concentration by centrifugation in Amicom filtering tubes to reach the desired concentration.

This process has been significantly improved by exchanging the fibrin polymer at concentration of 40 mg/ml through a dynamic dialysis system that significantly reduces exchange time against acetic acid, and avoids centrifugation to concentrate the monomer in solution.

Polymerization/Adhesion. The composition of solutions, method of production, and method of application (physicochemical characteristics of the fibrin monomer solution, composition of the neutralization buffer, and crosslinking of the polymer), which specifically refer to fibrin II and significantly differs from Edwardson, are critical to the performance of the fibrin polymer as a hemostatic agent. The hemostatic efficacy of sprayed film depends on the characteristics of fibrin itself (thickness of the fibers, the number of branch points, the porosity, and permeability), which result in a faster polymerization rate and stronger gel strength that is critical for the application. The invention creates opaque matrices of thick fibers, and therefore tube formation proceeds at a faster rate than in transparent matrices [5] (EXAMPLE 9). The described concentrations, dilutions and pH established for the Invention produce functional sealant with optimal fibrin structure, at a cost-effective concentration and at an accelerated rate.

The fibrin gel that seals the wound by stimulating the formation of a strong blood clot is formed as a result of the almost instant polymerization (within 5 seconds) of fibrin monomer solution by neutralization with a buffer, which in contact with the blood, wraps agglomerated and blood cells in covalent fibrin structure. Factor XIII present in the blood facilitates the transglutaminase-mediated oligomerization of the aC-domains of fibrin promoting integrin clustering and thereby increasing cell adhesion and spreading, which stimulates fibrin to bind avb3-, avb5- and a5b1-integrins on EC (9). The oligomerization also promotes integrin-dependent cell signaling via focal adhesion kinase (FAK) and extracellular signal-regulated kinase (ERK), which results in an increased cell adhesion further promoted by the fibronectin present in the fibrinogen, and cell migration [10]. The blood clot thus formed is mechanically stable, well integrated into the wound and more resistant to lysis by plasmin compared with an uncross-linked clot [8], or partially crosslinked clot of other fibrin sealants.

The adhesion characteristics to vital human tissue and the kinetics of polymerization of the proposed agent have been tested in vitro and in vivo. The data obtained and described in EXAMPLE 11 provide ample evidence of the ability of the Invention to stop diffuse arterial bleeding and achieve hemostasis without compression in induced burn wounds, and as adjunct in intraperitoneal light to moderate bleeding in soft tissue, solid organ and vascular wound repair.

### Invention Presentations:

The fibrin spray can be presented in several dosages, such as typical commercially desired volumes ranging from 4 ml to 20 ml, or greater, in a double aerosol can as described in FIGS. 1a to 3c, or in double-barrel syringes for dosages of 1 ml to 10 ml (FIG. 4). The application device is designed to be re-used in the course of the surgical period and discarded once the surgical procedure in concluded.

### SUMMARY OF THE INVENTION

The present invention lies within the domain of biological tissue sealants and hemostats, which are biodegradable and nontoxic, intended for therapeutic use, for example, as an adjunct to general hemostasis, as glue in opthalmological surgery or to debride necrotic surface of burns.

In one aspect, the present invention relates to a biocompatible adhesive fibrin polymer, which is bio-reabsorbable and nontoxic, for surgical or therapeutic use. In another aspect, the invention relates to a process for producing and applying a hemostatic spray.

Extensive in vivo studies show that ClotSpray is an excellent general hemostatic agent candidate for use as adjunct to general hemostasis in light to moderate bleeding. The agent is durable, easy to store, fast to prepare, poses minimal risk, requires little training to use, and is highly effective.

### BRIEF DESCRIPTION OF THE DRAWINGS

FIG. 1a a perspective view of the present invention, a double aerosol can assemblies to a central manifold that sprays the mixed biomaterial;

FIG. 1b is a side plan view of the present invention,

FIG. 1c is a top view of the present invention;

FIG. 2 a is a is a side view of the present invention with aerosol assembly; syringe attached to the valve inside the aerosol can

FIG. 2b is a side close up view the mounting of the valve to the aerosol can;

FIG. 2c is an exploded side view of the aerosol assembly

FIG. 3a is an exploded perspective view of the present invention; aerosol assembly;

FIG. 3b is a side view with hidden lines of the present invention;

FIG. 3c is a close up side view with hidden lines of the aerosol assembly attachment to the manifold

FIG. 4 Double barrel syringe applicator

FIG. 5a Dynamic dialysis membrane and tube description

FIG. 5b Dynamic dialysis flow diagram

FIG. 5c Dynamic dialysis perspective view of system

FIG. 6. rheometry measurements of fibrin monomer in acetic acid and glycine solution, pH 3.5

FIG. 7. Rheometry measurements of polymerized fibrin monomer (FC) with a concentration of 19, 30, and 45 mg/mL and fibrinogen cleaved by thrombin (FS) at a concentration of 40 mg/mL.

FIG. 8. rheometry measurements of polymerized fibrin monomer (FC) with 19, 30, and 45 mg/mL, and fibrinogen cleaved by thrombin (FS) at a concentration of 40 mg/mL. created on the surface of freshly drawn canine blood

FIG. 9. Analysis of the sedimentation profiles of all three samples, Fgn-1, Fib-2 and Fib-3, by ultracentrifugation.

FIG. 10. Thermal denaturation (unfolding) of fibrin monomer in 50 mM Glycine buffer at pH 3.5 (black), pH 3.0 (blue), and 2.5 (red) detected by Circular Dichroism measurements

FIG. 11a SDS-PAGE of polymerization of fibrin monomer stored for 0 days

FIG. 11b SDS-PAGE of polymerization of fibrin monomer stored for 7 days

FIG. 11C SDS-PAGE of polymerization of fibrin monomer stored for 150 days

FIG. 12 A HPLC chromatograms for fibrin monomers

FIG. 12 B HPLC chromatograms protein standards

FIG. 13a Clots prepared with CSL fibrin Ph 4.0 analyzed by scanning electron microscopy at 10,000 magnifications

FIG. 13b Clots prepared with fibrin Ph 4.0 analyzed by scanning electron microscopy at 10,000 magnifications

FIG. 13c Clots prepared with CSL fibrin Ph 3.5 analyzed by scanning electron microscopy at 10,000 magnifications

13d Clots prepared with fibrin Ph 3.5 analyzed by scanning electron microscopy at 10,000 magnifications

FIG. 14 a Analysis by SDS-PAGE of fibrin cross-linking by Factor XIII and calcium independent transglutaminase enzyme at 1, 5 and 10 Minutes.

FIG. 14.b Analysis by Western blot of fibrin cross-linking by Factor XIII and calcium independent transglutaminase enzyme at 1, 5 and 10 Minutes.

FIG. 14c Rheometry measurements of polymerized fibrin monomer in the presence of calcium independent transglutaminase enzyme as compared to Factor XIII

FIG. 15a Biodegradation Liver 100× Biopsy area UV light day 31

FIG. 15b Biodegradation Liver 400× Biopsy area UV light day 31

FIG. 15c Biodegradation 15 c Liver Positive Control

FIG. 16 a Biodegradation Liver 100× Negative control UV light day 31

FIG. 16 b Biodegradation Liver Background 100×

FIG. 17 a Preparations tested for biocompatibility with human epithelial cells Untreated day 5

FIG. 17 b Preparations tested for biocompatibility with human fibroblasts ClotSpray day 5

FIG. 18 a Preparations tested for biocompatibility with human epithelial cells (A549 cell line, ATCC) Untreated, Day 5

FIG. 18 b Preparations tested for biocompatibility with human epithelial cells (A549 cell line, ATCC) ClotSpray, Day 5

FIG. 19 Manufacturing flow chart.

### DETAILED DESCRIPTION

We have developed an aerosolized hemostatic agent comprising a two-part solutions, the Invention currently called ClotSpray, for use as an adjunct to general hemostasis in moderate hemorrhage during surgical procedures, and in treatment of burn wounds, and as fibrin glue to bond tissues or membranes to tissues. The composition can also be used in a double barrel syringe similar to current fibrin sealant application methodologies. The Invention is a fibrin-hemostatic glue depleted of thrombin, with no collagen or polymer support, designed to create hemostasis through the formation of a stable fibrin clot with or without compression.

The Invention consists of a fibrin monomer dissolved in acetic acid solution at pH 3.2-3.8, which is produced by an improved method of dialysis manufacturing process over the one described in U.S. Pat. No. 8,367,802. The fibrin monomer is neutralized by a neutralization buffer solution composed of:

Buffer A For mixing of 1 part of fibrin monomer with 1 part of neutralization buffer

### 100 mM HEPES

### 300 mM NaCl

### 20 mM CaCl₂

pH adjusted to 7.5

Buffer B For mixing of 3 parts of fibrin monomer with 1 part of neutralization buffer:

### 300 mM HEPES

### 1200 mM NaCl

### 40 mM CaCl₂

pH adjusted to 8.2
and Calcium independent Transglutaminase enzyme (ACTIVA) which can be dissolved into the buffer or incorporated in dry form within the fluid path for longer stability.

The fibrin polymer resulting from the neutralization of the monomer is further crosslinked by a reaction with Factor XIII from the blood.

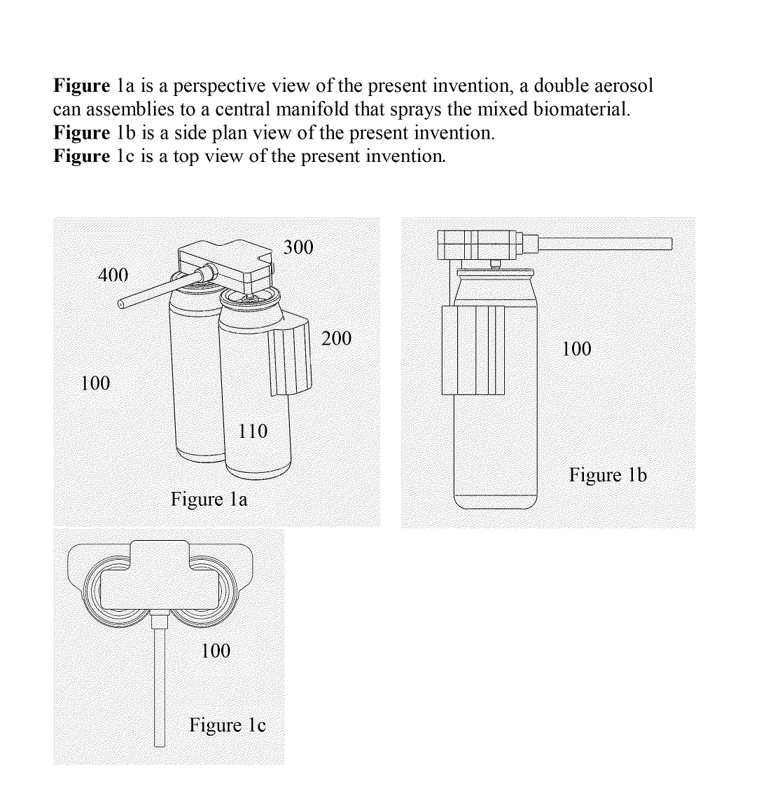

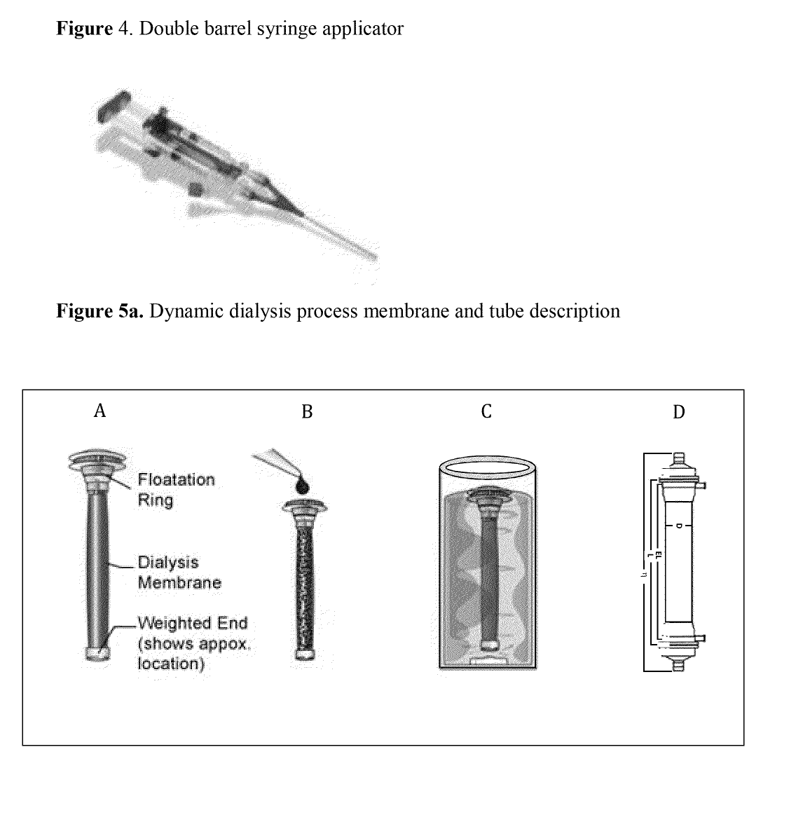

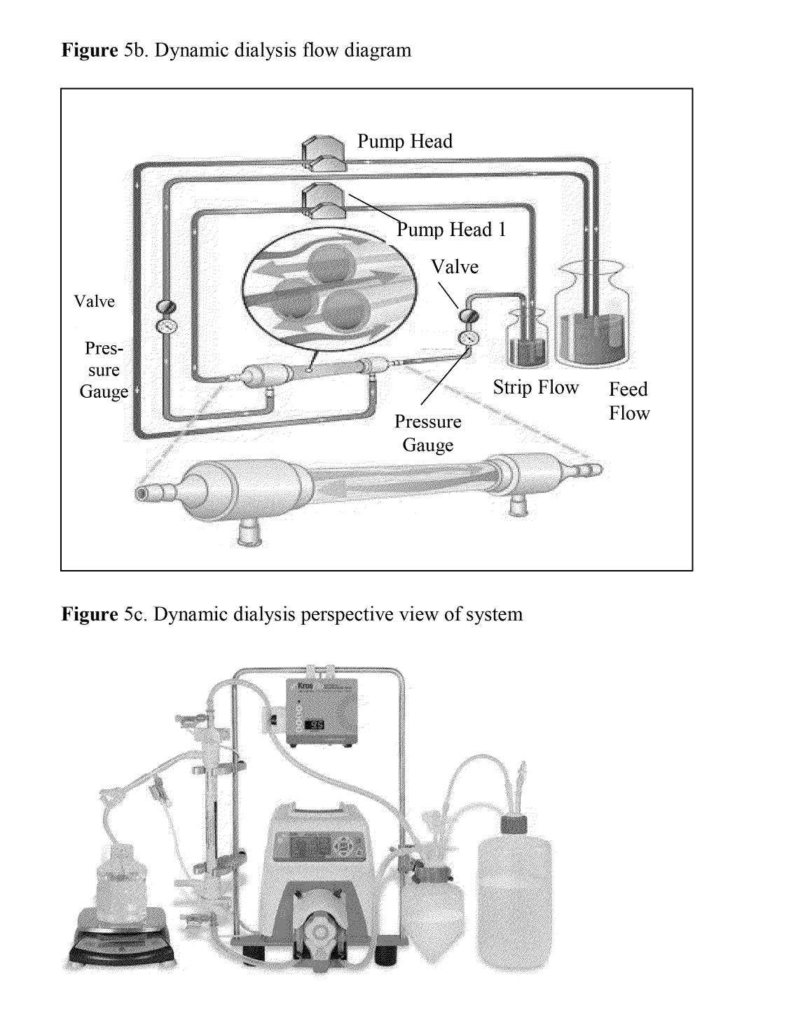

The sprayed fibrin layer is applied by an aerosol as described in FIGS. 1a, 1b, 1c, 2a, 2b, 2c, 3a, 3b, 3c, or a double-barrel syringe FIG. 4, over lacerated bleeding tissue, forming a sticky, semi-cohesive gel barrier and subsequently a fibrin clot. The agent seals the wound with a blood clot within 3-5 seconds. Each solution is sterilized by filtration through a 0.22 micron Millipore filter.

### Preparation of Fibrin Monomer Solution.

To best understand the present invention, the difference between fibrin I and fibrin II needs to be appreciated (see attached published paper by Medved and Wiesel). To produce fibrin II, which is also called desAB-fibrin, thrombin (this is a plasma-derived enzyme) removes two pairs of fibrinopeptides A and B resulting in the exposure of two pairs of polymerization sites that are responsible for the formation of strong fibrin II polymers, which unlike fibrin I is hard. In contrast, thrombin-like enzymes remove from fibrinogen only a pair of fibrinopeptides “A” resulting in the exposure of only one pair of polymerization sites. Because the second pair of polymerization sites is not exposed or activated, such fibrin, called fibrin I or desA-fibrin (also called “soft” fibrin), is less strong than fibrin II and can be easily dissolved in acid solution.

Applicants developed their own patentable methodology for preparation of a fibrin II monomer solution, as described U.S. Pat. No. 8,367,802 Falus et al. Monomer II in acetic acid solution at a concentration of 12 mg/ml as described in this patent is produced as follows: Three grams (3 gr.) of fibrinogen is dissolved in 600 ml of 20 mM HEPES buffer pH 7.3, 0.15 mM sodium chloride, and 5 mM calcium Chloride. A total of 15 ml of thrombin solution in HBS buffer at a concentration 50 NIH U/mL is prepared. The fibrinogen and thrombin solutions are gently mixed in a dialysis tube to make homogeneous solution. This mixture is incubated at 37° C. in a glass jar containing HBS buffer, using either water bath or incubator for 2 hr. After the incubation, the dialysis tubes are immersed in Acetic Acid solution for 1 hr. at 4° C.; with gentle stirring to exchange the reaction buffer. This process is repeated twice. Following three one-hour dialysis periods, the acetic acid solution is replaced by a fresh batch of acid and kept at 4° C. overnight with gentle stirring; to dissolve the clot inside the tubes. In the above method, the clear, cold acidic solution (˜pH 3.45) is concentrated from 4-5 mg/ml to 11-13 mg/mL using AMICON Ultra (30,000 MWCO, Millipore sterile) centrifugal filter devices at 3000 RPM in a refrigerated centrifuge at 4° C. This method is herewith improved, and the solution produced is characterized as “AC strength fibrin II monomer solution” in order to reduce the number of steps that results in a fibrin polymer with the viscoelastic characteristic necessary to control bleeding under a moderate flow of blood.

### Improved Method of Manufacture of Fibrin Monomer in Acid Solution

The improved method included in this invention creates a Fibrin-II polymer solution or desAB fibrin polymer solution at a higher concentration (40 mg/ml) than described in U.S. Pat. No. 8,367,802, which can be dissolved faster by dynamic dialysis in a way compatible with industrial production (8 hours) without loss of protein, and if needed can be rapidly concentrated by centrifugation up to 60 mg/ml.

The first step encompasses the polymerization of fibrinogen with thrombin to produce fibrin II polymer, which is then dissolved into fibrin II monomer by dynamic dialysis against acetic acid, with pH adjusted to 3.5. For the preparation of AC Strength fibrin II monomer in acid solution, fibrinogen is dialyzed against glycine buffer first, cleaved with thrombin and then, the resulting polymer is dialyzed against acetic acid. More specifically fibrinogen at the concentration of 40-50 mg/ml is dialyzed in dynamic dialysis tube against glycine buffer (20 mM glycine, 450 mM sodium chloride) pH 8.5 overnight at 4° C. Thrombin solution (100 NIH U/ml) is added to the dialysis tubes containing fibrinogen and incubated for 3 hours. The fibrinogen to thrombin ratio is 50 ml:1 ml. The solution is then dialyzed in the dynamic dialysis system against acetic acid pH 3.5, until the white polymer is completely dissolved, (1 hr to 3 hours) and the thrombin used for converting fibrinogen into fibrin polymer is removed. The fibrin is then collected, the concentration is measured and diluted or concentrated up to 60 mg/ml as desired concentration. This production method incorporates a sterilization step protocol in which the monomer solution is filtered through 0.22 μM filter and stored at 2-8° C. in the Millipore sterilized pyrogen-free container until filled into syringes. Such dialysis results in complete solubilization of fibrin II monomer and its further purification from contaminating plasma proteins including thrombin and plasmin(ogen).

The dialysis process in a one way membrane contained within a dynamic dialysis system, described in FIG. 5a, 5b, 5c, breaks the polymer bonds and produces a Fibrin II monomer in acid solution. This process is required since the fibrin II polymer cannot be easily nor efficiently converted to a fibrin II monomer by simple dissolution in acetic acid (as proposed by Edwardson).

Applicants' use dialysis and storage of fibrin monomer II in acetic acid, with pH 3.2 to 3.8 and preferentially at 3.5, which has a concentration of about 0.125%. At this concentration, fibrin monomer preserves its native structure and activity and can form fibrin polymer upon its transfer to neutral pH (pH 7.0).

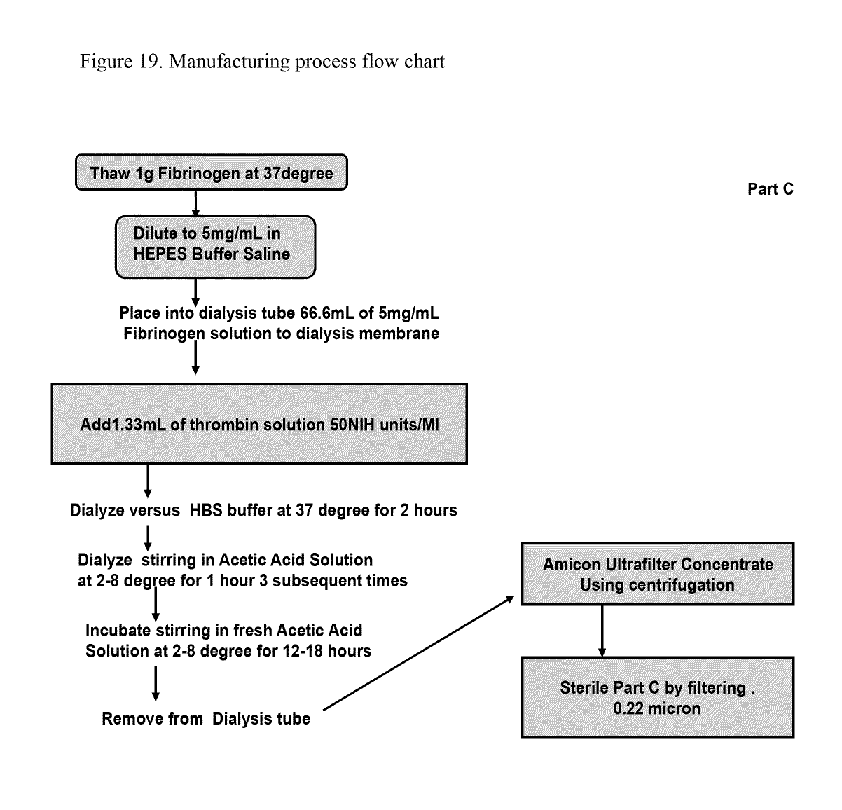

Applicant has chosen to store the monomer at pH 3.2-3.8 following a turbidity assay establishing experimentally that fibrin polymerization from pH 3.5 creates a stronger fibrin polymer with thicker fibers. EXAMPLE 9. The manufacturing process (flow chart) is described in FIG. 19.

### Detailed Description of the Dynamic Dialysis Method

Dynamic Dialysis uses flow dynamics to increase both the rate and efficiency of dialysis. Circulating the sample and/or the dialysate creates the highest possible concentration gradient and generates a pressure differential to significantly decrease dialysis time. This supplemental driving force increases the hypo-osmotic mass transfer rate across the semi-permeable membrane and allows for sample concentration during the dialysis process. The invention uses a system that recirculates the exchange buffer within a tube containing a membrane with a pore size of 100 kD. The system increases by 400% the dissolution rate of the fibrin polymer.

The dialysis module consists of an open-ended dialysis membrane potted in a tubular housing to create two distinct flow chambers, lumen and extracapillary, each with inlet and outlet port access. The semi-permeable dialysis membrane that separates the two chambers selectively permits passage based on size and concentration gradient of solutes while restricting other solutes from passing between the 2 chambers. By operating the module in a counter-current flow mode, the solutes passing through the membrane are quickly swept away and diluted into a large volume of dialysate solution, maintaining the largest concentration gradient possible. (FIGS. 5a, 5b, 5c) With this method Fibrin II Polymer is completely dissolved in acetic acid (Yield 99.6%) within 3 hours allowing for a battery of 60 dialysis columns with standard commercially available membranes to produce 40,320 MI of monomer with a clottability of 99.7%

### Detailed Description of the Aerosol-Like Applicator

Embodiments of the present invention are described herein in the context of a biomaterial applicator using modified aerosol can and valve technology, aerosol pressure-induced syringe displacement for ejection of biomaterial(s), and manifold apparatus for mixing of a plurality of biomaterial components delivered from the aerosol apparatuses. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same reference indicators will be used throughout the drawings and the following detailed description to refer to the same or like parts.

The present invention comprises a double aerosol can, a modified aerosol valve and a syringe with a floating syringe piston that is attached to the aerosol valve by a luer-lock connection or similar. An aerosol can holder attaches a plurality of aerosol can assemblies to a central manifold that receives the biomaterial(s) from the aerosol can assemblies. A spring mechanism in conjunction with the manifold assembly and the aerosol can holder is designed to actuate the aerosol valves, and a biomaterial mixing applicator. These components work in conjunction to deliver biomaterial into a patient's body during a surgical procedure, where high pressurized displacement of the biomaterials contained within the syringes aids in dispersing the biomaterial in a spray fashion, but without any compressed gas included within the biomaterial spray delivery. When the valve is actuated and the pressure within the can is exposed to atmospheric pressure, the high pressure within the aerosol can will then displace the floating plunger within the syringe, and eject the biomaterial out of the valve into the mixing manifold.

In the preferred embodiment, the biomaterial is contained within a glass or biocompatible polymer syringe with a floating piston. The syringe is then attached to an aerosol valve, which has been modified to connect to a luer-lock syringe. It is noted that other product containers embodiments such as a Bag-on-valve technology could also be used in place of a conventional syringe with all other subsequent components of the invention working the same. The aerosol valve attached to the syringe is mounted within a mounting cup and attached to the aerosol can. The aerosol can is charged with a pressurized gas such as carbon dioxide, nitrogen, or air. The aerosol can(s) are then affixed inside an aerosol can holding apparatus. A central manifold assembly is then positioned over the aerosol cans. The stems of the aerosol valves are concentrically aligned with receiving channels of the manifold assembly. A spring mechanism either solely from the springs inside the aerosol valve, or and additional exterior spring provide interaction between the manifold and the aerosol can holding apparatus. The actuation mechanism that depresses the stem inside the valve will initiate delivery of the biomaterial. When the aerosol valve is opened and exposed to atmospheric pressure, the pressure within the can then displaces the floating piston within the syringe containing the biomaterial(s). The biomaterials are ejected from the syringe with the appropriate force and displacement rate into the valve and ultimately into the manifold assembly. The charging pressure within the can could be optimized to obtain the desired spray pattern of the biomaterial based upon the properties of the biomaterial as well as other distal pressure drop considerations. Further, different flow rates can be achieved between the aerosol cans to mix the biomaterials in different proportion (i.e. 3:1 ratio of fibrin to neutralization buffer). Finally, a biomaterial applicator can then be affixed to the manifold assembly to mix the plurality of biomaterial components and provide a spray delivery of the biomaterial to the surgical site. The biomaterial applicator can also include various enhanced internal mixing elements such as static mixers, or mechanical breakup units if the two or more biomaterials are of a viscous nature requiring such enhanced mixing elements.

The invention also supports the ability to connect multiple individual aerosol cans, containing different components of a biomaterial with their respective individual syringes The illustrations of this patent will present two aerosol can assemblies for a dual component biomaterial, but any number of aerosol can assemblies could be employed to mix biomaterials that contain more than two components. The manifold assembly would be designed to accept as many aerosol can assemblies as necessary.

These and other objectives of the present invention will become obvious to those of ordinary skill in the art after reading the following detailed description of the preferred embodiments. It is to be understood that the foregoing general description and the following detailed description are exemplary, and are intended to provide further explanation of the invention as claimed. Embodiments of this invention may best be understood by referring to the following description and accompanying drawings that are used to illustrate the invention. In the drawings:

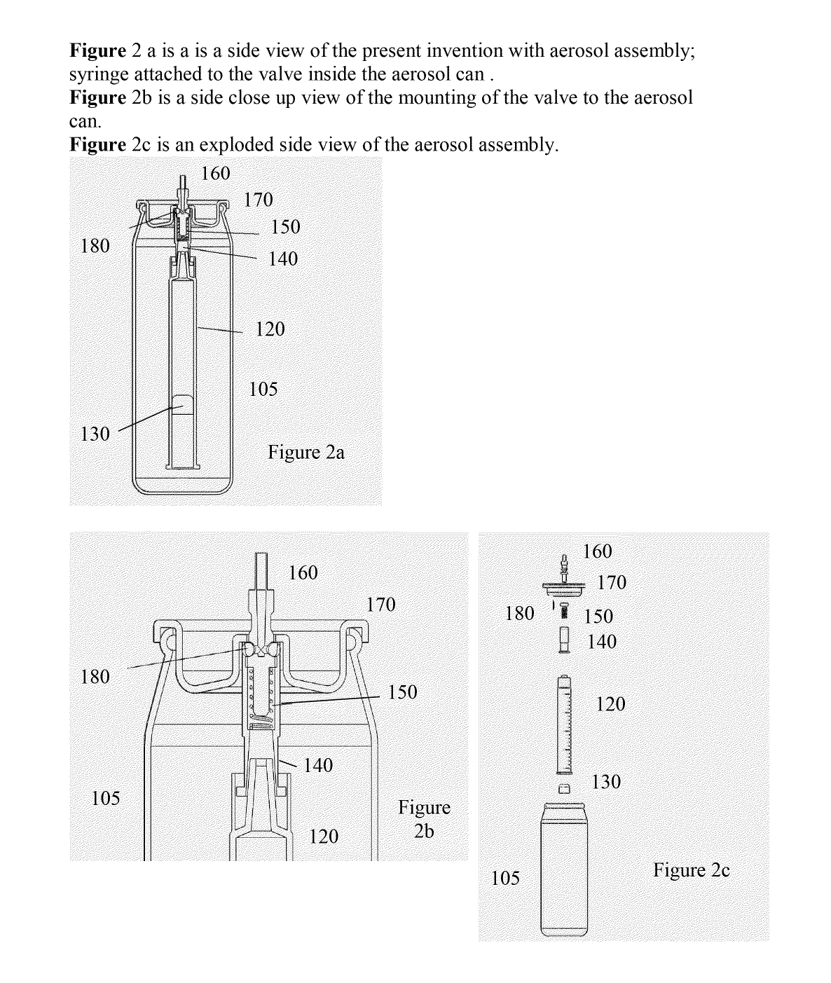

Now referencing the figures, FIG. 1a discloses the biomaterial aerosol ejected mixing and spray system 100, comprising an aerosol assembly 110, aerosol can holder 200, manifold assembly 300, and biomaterial applicator 400. FIGS. 1b, and 1c illustrate the biomaterial aerosol ejected mixing and spray system 100 in greater detail. FIG. 2a illustrates the components of the aerosol assembly including the aerosol can 105, the biocompatible syringe containing the biomaterial 120, the floating syringe piston 130, the valve body with luer-lock syringe feature attachment 140, the valve spring 150, the valve stem 160, the mounting cup for the valve assembly 170, and the valve stem gasket 180. This aerosol assembly 110 ejects the biomaterial into the manifold assembly 300. FIGS. 2b, and 2c illustrate the components of the aerosol assembly in greater detail.

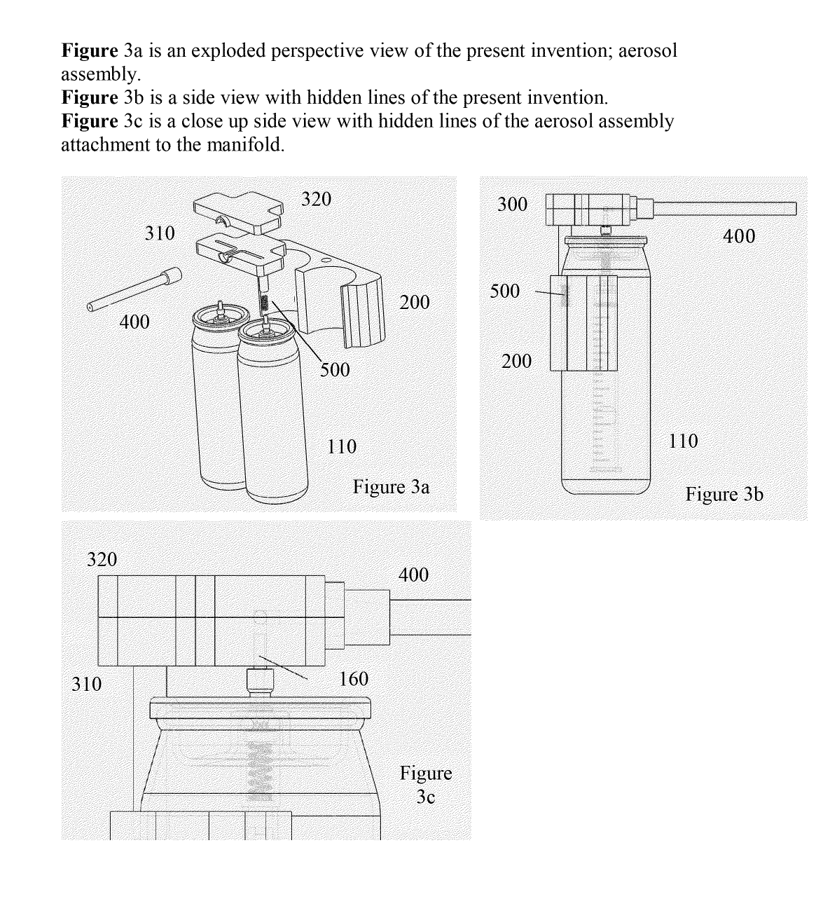

FIG. 3a illustrates the biomaterial aerosol delivery, mixing and spray system 100 in exploded view. FIGS. 3a, and 3b Illustrate the biomaterial aerosol delivery, mixing and spray system 100 in greater detail.

FIG. 4a illustrates the manifold assembly 300 consisting of an upper manifold component 320, a lower manifold component 310, and a spring 500. FIG. 4b illustrates the interaction between the manifold assembly 300 and the aerosol can holder 200. The spring 500 is seated in the core of the aerosol can holder 200. When the user presses down on the manifold assembly 300, the spring 500 provides actuation feedback, and the lower manifold component 310 will press on the aerosol valve stem 160 resulting in ejection of the biomaterial into the manifold assembly 300. The biomaterial(s) will then be introduced into a biomaterial mixing applicator 400, where they will be ejected from the applicator 400 in a spray output to the surgical site.

### EXAMPLES

### Example 1

### Studies to Determine the Viscoelastic Properties of the Fibrin Clot Resulting from Variations on Buffer Composition (Process)

Purpose: To compare viscoelastic properties by rheometry of fibrin clots with fibrin monomers in acetic acid or glycine solutions at pH 3.5.

Materials: Fibrin monomers at 40 mg/mL in acetic acid, pH 3.5, or glycine buffer, pH 3.5 and neutralization buffer were used for this study.

Method: G′ “rheometry” of both types of fibrin clot were measured using Carri Med Rheometer, TA instruments, model CSL²₅₀₀. Neutralization buffers designed to create fibrin clot from fibrin in acetic acid or glycine were mixed with fibrin in volume ratio 1 to 1. All measurements were done in duplicates.

Results:

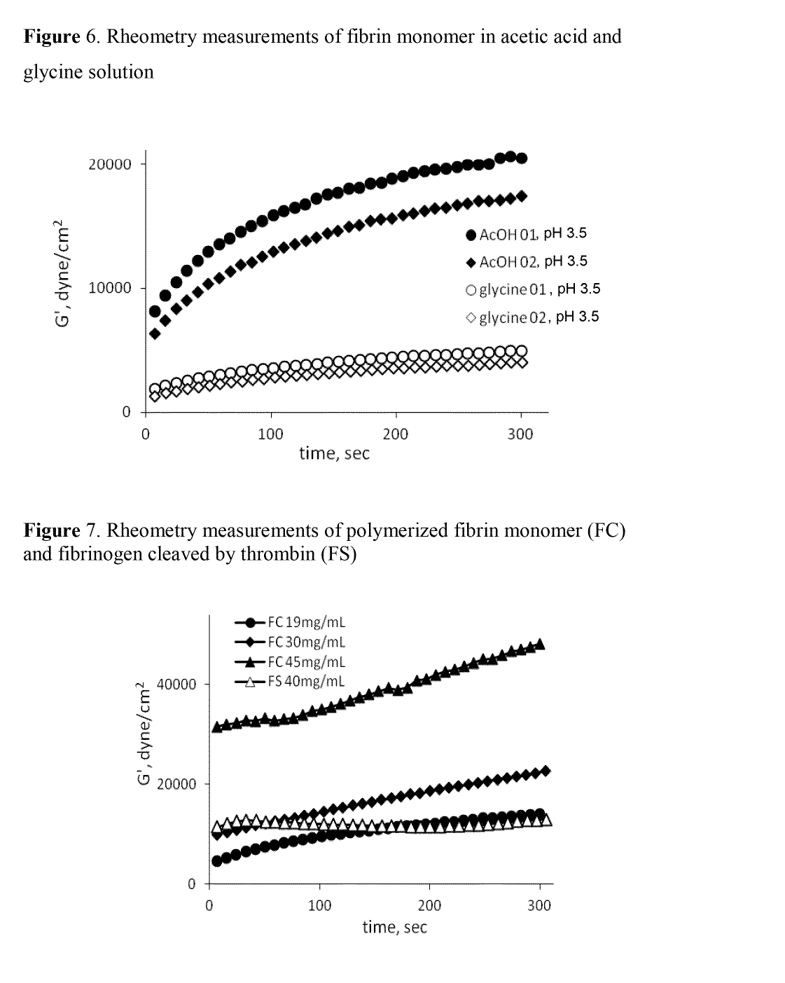

The results rheometry measurements of fibrin monomer in acetic acid and glycine solution, pH 3.5, are presented in FIG. 6

Conclusions:

Rheometry data of fibrin monomer with different buffers demonstrated significantly higher G′ values of fibrin clot with acetic acid solution, pH 3.5.

### Example 2a

### Comparison of Viscoelastic Properties of a Fibrin Clot Produced by Neutralization of ACStrength Fibrin Monomer II Solution Vs Cleavage of Fibrinogen by Thrombin

Purpose: To evaluate the strength of the fibrin gel produced by neutralization of fibrin monomer solution Vs standard fibrin sealant by rheometry.

Materials: Fibrin monomer solutions at concentrations of 25, 40, and 60 mg/mL. Fibrinogen at 80 mg/mL and thrombin at 500 IU/mL. Neutralization buffers composed of HEPES, sodium chloride and calcium chloride

Method: G′ rheometry measurements in sheer stress mode of fibrin gels polymerized on the rheometry plate by neutralization of fibrin monomer solution, and cleavage of fibrinogen by thrombin or fibrin sealant (FS) was measured according to rheology protocol using Carri Med Rheometer, TA instruments, model CSL²₅₀₀. Fibrin monomer at 25, 40, and 60 mg/mL was mixed with neutralization buffer in volume ration of 3 to 1 to produce a fibrin polymer (FC) containing 19, 30, or 45 mg/mL of fibrin. Fibrinogen solution at a concentration of 80 mg/ml was mixed with thrombin solution at a concentration of 500 u/ml in volume ration 1 to 1 to produce a fibrin polymer (FS) at a concentration of 40 mg/ml.

Results:

The results of rheometry measurements of FC with a concentration of 19, 30, and 45 mg/mL and fibrin polymer produced by cleavage of fibrinogen by thrombin (FS) with a concentration of 40 mg/mL of fibrin are presented in FIG. 7.

Conclusions:

G′ Rheometry data shows that FC at a final concentration of 19 mg/ml has a similar gel strength than FS at a concentration of 40 mg/ml. At similar fibrin concentrations the gel strength of FC is 100% higher in dyn/cm2 values than FS. (fibrinogen cleaved by thrombin).

### Example 2b

### Comparison of Viscoelastic Properties by Rheometry of a Blood Clot Produced by ACStrength Monomer II Solution at Various Concentrations Against Standard Fibrin Sealant

Purpose: To compare viscoelastic properties of fibrin polymer created by polymerization of fibrin monomer solution Vs. fibrin sealant produced by cleavage of fibrinogen by thrombin on a surface of fresh blood placed on the rheometer plate.

Materials: Fibrin monomers at 25, 40, and 60 mg/mL. Fibrinogen at 80 mg/mL and thrombin at 500 IU/mL. Neutralization buffers. Freshly drawn canine blood.

Method: G′ “rheometry” of a blood clot created by polymerization of fibrin monomer solution (FC) and by a fibrin sealant (FS) produced by cleavage of fibrinogen by thrombin was measured according to rheology protocol using Carri Med Rheometer, TA instruments, model CSL²₅₀₀. Fibrin monomer at 25, 40, and 60 mg/mL was mixed with neutralization buffer in volume ratio 3 to 1 to create FC containing 19, 30, or 45 mg/mL of fibrin. To create FS with 40 mg/mL of fibrin, fibrinogen solution at the concentration of 80 mg/ml was mixed with thrombin solution in volume ratio 1 to 1. FC and FS were applied on a surface of freshly drawn canine blood before measurements.

Results:

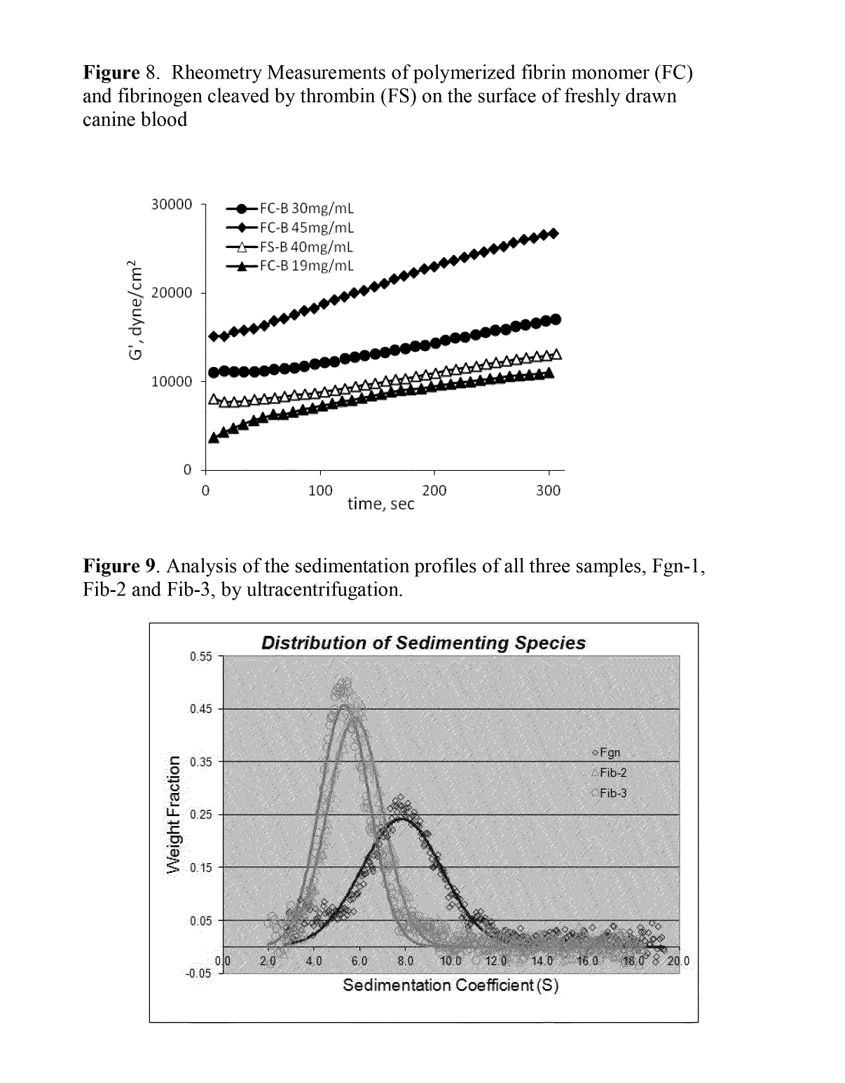

The results rheometry measurements of FC with 19, 30, and 45 mg/mL of fibrin and FS with 40 mg/mL of fibrin created on the surface of freshly drawn canine blood are presented in FIG. 8.

Conclusions:

Rheometry data of FC and FS created on the surface of freshly drawn canine blood demonstrated that the polymerization of fibrin monomer solution produces a stronger blood clot than fibrinogen cleaved by thrombin.

### Example 3

### Characterization of Fibrin Monomer Solution in Terms of Protofibrils Aggregate and Degradation Products as Compared to Fibrinogen Solution

Materials and Methods: Sedimentation velocity experiments by analytical ultracentrifugation were performed by Dr. Roy Hantgan, Wake Forest University, School of Medicine—Molecular Partners.

Fibrinogens were obtained from two sources; (GR) and CSL Behring (CSL). HEPES buffer; pH 7.5 with 150 mM NaCl, 2.5 mM CaCl₂ (HBS); Neutralization buffer for fibrin monomer at pH 3.5 (NB 3.5), prepared by Biomedica Nov. 4, 2013; Neutralization buffer for fibrin monomer at pH 4.0 (NB 4.0), prepared by Biomedica Nov. 4, 2013; Activa (Part D): 0.3 g/mL in 10 mM HEPES, pH 8.2 with 100 mM CaCl₂; prepared by Biomedica Nov. 4, 2013; Factor XIII (FXIII) at 4700 U/mL from Enzyme Research Laboratories; Thrombin (Thr) at 500 NIH U/mL. Fibrin monomers were prepared on Nov. 4, 2013: 25 mg/mL of GR fibrin at pH 4.0, 25 mg/mL of CSL fibrin at pH 4.0 (fibrinogen source—CSL Behring), 25 mg/mL of CSL fibrin at pH 3.5 (fibrinogen source—CSL Behring).

Fibrinogen sample (Fgn-1) was prepared by overnight dialysis of fibrinogen at 0.67 mg/ml versus HBS buffer, pH 7.4. Fibrin monomer samples were prepared by overnight dialysis of fibrin monomer at 0.67 mg/ml versus 50 mM Gly buffer, pH 3.5 (Fib-2) or acetic acid, pH 3.5 (Fib-3). Samples centrifugation was done on a Beckman Optima XL-A analytical ultracentrifuge (Beckman Instruments, Palo Alto, Calif.) equipped with absorbance optics and an An60 Ti rotor according to protocol described in [Tsurupa et al., 2004, Biophys. Chem. 112: 257-266; see attached]. Sedimentation velocity data were analyzed using both SVEDBERG (version 1.04) and DCDT+ (version 6.31) software (J. Philo, Thousand Oaks, Calif.) to obtain the weight-average sedimentation coefficient (S_{w}) and distribution of sedimenting species.

Results: Analysis of the sedimentation profiles of all three samples, Fgn-1, Fib-2 and Fib-3, shown in FIG. 9 revealed that they all behaved as single species with sedimentation coefficients (S_{w}) equal to ˜8 S, ˜6 S, and ˜5.5 S, respectively, as determined by fitting the radial distribution data with SVEDBERG. No larger species (dimers or higher molecular weight aggregates) were observed in all three samples. Fibrinogen sample Fgn-1 exhibited the expected ˜8 S value while both fibrin samples sedimented slower, most probably due to the primary charge effect, i.e. because charged macromolecules in solvents of low salt concentration display sedimentation coefficients lower than that measured in isoelectric solutions, as described in [Introduction to Analytical Ultracentrifugation, Beckman; see attached].

Analytical Ultracentrifugation experiments confirmed that all three samples, namely, fibrinogen (Fgn-1), fibrin monomer in glycine buffer (Fib-2), and fibrin monomer in acetic acid (Fib-2), were monomeric in solution; no significant amount of larger species (dimmers, aggregates) or degradation products were observed.

### Example 4

### Clotting Time Comparing Fibrin Monomer Polymerization Rate to a Fibrin Sealant

Purpose: To establish the rate of polymerization of ACstrength fibrin II solution when neutralized by a neutralization buffer as compared the rate of polymerization of fibrinogen cleaved by thrombin.

Materials and Methods: For the cleavage of fibrinogen by thrombin, fibrinogen at a concentration—8%, was diluted up to 12 mg/mL with HBS-Ca⁺² buffer (20 mM HEPES, pH 7.3, 150 mM NaCl, 2.5 mM CaCl₂) and this solution was used for Clotting Time and Clottability. Thrombin (activity—500 IU) was thawed at room temperature and kept at 2-8° C. before use. Activated factor XIII (Enzyme Research Lab, concentration—7.11 mg/mL, activity—2039 Loewy U/mg, was used for SDS page test.

For the cleavage of fibrinogen 250 L of fibrinogen at the concentration of 12 mg/mL was mixed with 750 L of HBS-Ca⁺² buffer containing 1 U/mL of thrombin.

Fibrin monomer solution was prepared as described above. Collected fibrin monomer solution was diluted up to 12 mg/ml and used to determine Clotting Time and Clottability.

Clotting time and Clottability: Clotting time was determined by measuring the formation of the polymer visually. Clottability was determined by the absorbance by spectrophotometer of the liquid squeezed out the formed polymer.

Results: The average clotting time of fibrinogen at 12 mg/ml cleaved by thrombin was 37 seconds. The clottability of resulted gel was 99.78%.

The average clotting time of neutralized fibrin monomer solution at 12 mg/ml was 12 seconds. The clottability of resulted polymer was 99.98%.

Conclusion: The kinetics of polymerization of the fibrin monomer solution tested in laboratory studies showed that neutralization of ACstrength fibrin II solution forms a fibrin polymer within 12 second of mixing it to neutralization buffer or 3 times faster than cleavage of fibrinogen by thrombin at the same concentration. Clottability of both samples was in the same range.

### Example 5

### Variation in Clotabilitty, Polymerization, and Clotting Time Based on pH the Purpose of these Experiments is to Demonstrate that Fibrin Monomer can Preserve its Native Structure and Clotting Properties Only in a Narrow pH Range, Namely, at pH 3.0-4.2, and that Polymerization Time is pH Dependant within this Range

Past experiments performed by differential scanning calorimetry (Privalov and Medved, 1982, J. Mol. Biol. 159: 665-682; Medved et al., 1982, FEBS Letters 146: 339-342) revealed that thermal denaturation (unfolding) of fibrin(ogen) domains containing polymerization sites is pH sensitive and irreversible. Namely, at pH 3.5 denaturation transition of fibrinogen starts at about 35° C. with midpoint of transition at 45.8° C. (FIG. 7 and Table 1 in: Privalov and Medved, 1982, J. Mol. Biol. 159: 665-682) and at pH 2.8 denaturation starts at about 18° C. with a midpoint of transition at 30° C. (FIG. 1 in: Medved et al., 1982, FEBS Letters 146: 339-342). Thus, because the denaturation is irreversible, incubation of fibrin(ogen) at room temperature in a solution having pH value of 2.8 should result in its complete denaturation and loss of the ability to form normal fibrin polymer. To further reinforce the abovementioned statement, we have recently performed the following experiments.

### Experiment 5.1

Fibrin monomer at concentration of 13 mg/ml stored at pH 3.5 was divided into three portions and each portion was transferred to 50 mM Glycine buffers having pH 2.5 (sample 1), pH 3.0 (sample 2), and 3.5 (sample 3) by dialysis at 4° C. for 22 hours. The temperature-induced unfolding of each sample was then measured by monitoring Circular Dichroism (CD) changes using a Jasco-810 spectropolarimeter. The results presented in the FIG. 9 indicate that fibrin monomer at pH 3.5 exhibits a typical sigmoidal transition in a temperature range between 30 and 60° C. with a midpoint of the transition at about 45° C. The curves have been arbitrary shifted along vertical axis to facilitate comparison. The dashed lines represent linear extrapolation of the CD values before and after unfolding transitions to highlight their sigmoidal character; the vertical lines show midpoints of unfolding transitions. Please note that the transitions starting after 70° C. correspond to the unfolding of the central region of fibrin (HT transitions), as described in (Privalov and Medved, 1982, J. Mol. Biol. 159: 665-682).

Thus, thermal stability of fibrin monomer at this pH is essentially the same as that previously observed for fibrinogen at pH 3.5 (Privalov and Medved, 1982, J. Mol. Biol. 159: 665-682). In contrast, at pH 3.0 the transition occurred at lower temperature and its amplitude was lower suggesting partial denaturation. No unfolding transition was observed at pH 2.5 indicating complete denaturation.

### Experiment 5.2

Aliquots from each sample were taken for determination of clotting time (time at which fibrin polymer is formed) using our standard clotting assay. Namely, when 200 mkL of sample 1 (pH 3.5) and 200 mkL of sample 2 (pH 3.0) each were neutralized with 600 mkL HEPES buffer, pH 7.5, the clot was formed within 14 and 20 sec, respectively. This indicate that both sample preserved native structure although the extended clotting time of sample 2 suggests that at pH 3.0 fibrin monomer may be partially denatured. In contrast, when sample 3 (200 mkL, pH 2.5) was neutralized with 600 mkL HEPES buffer, pH 7.5, fibrin monomer immediately precipitated in solution indicating that the protein was already completely denatured.

### Experiment 5.3

Fibrin monomer at 20 mg/mL in acetic acid solution, pH 3.5, was dialyzed at 4° C. versus five buffers: (1) 50 mM Gly, pH 3.5; (2) 50 mM sodium acetate buffer, pH 4.0; (3) 50 mM sodium acetate buffer, pH 4.5; (4) 50 mM sodium acetate buffer, pH 4.8; (5) 50 mM sodium acetate buffer, pH 5.0. After overnight dialysis, samples 1, 2, and 3 (pH 3.5, pH 4.0, and pH 4.5, respectively) were transparent and exhibited similar clotting time, about 10 sec, while samples 4 and 5 (pH 4.8 and 5.0, respectively) exhibited strong precipitation. Further, when sample 3 (pH 4.5) was further kept in 50 mM sodium acetate buffer, pH 4.5, for additional 24 hours, a slight precipitate was observed indicating that at this pH the system is unstable. These experiments indicate that the upper limit of pH value at which fibrin monomer can be kept in solution is 4.0-4.2.

### Experiment 5.4

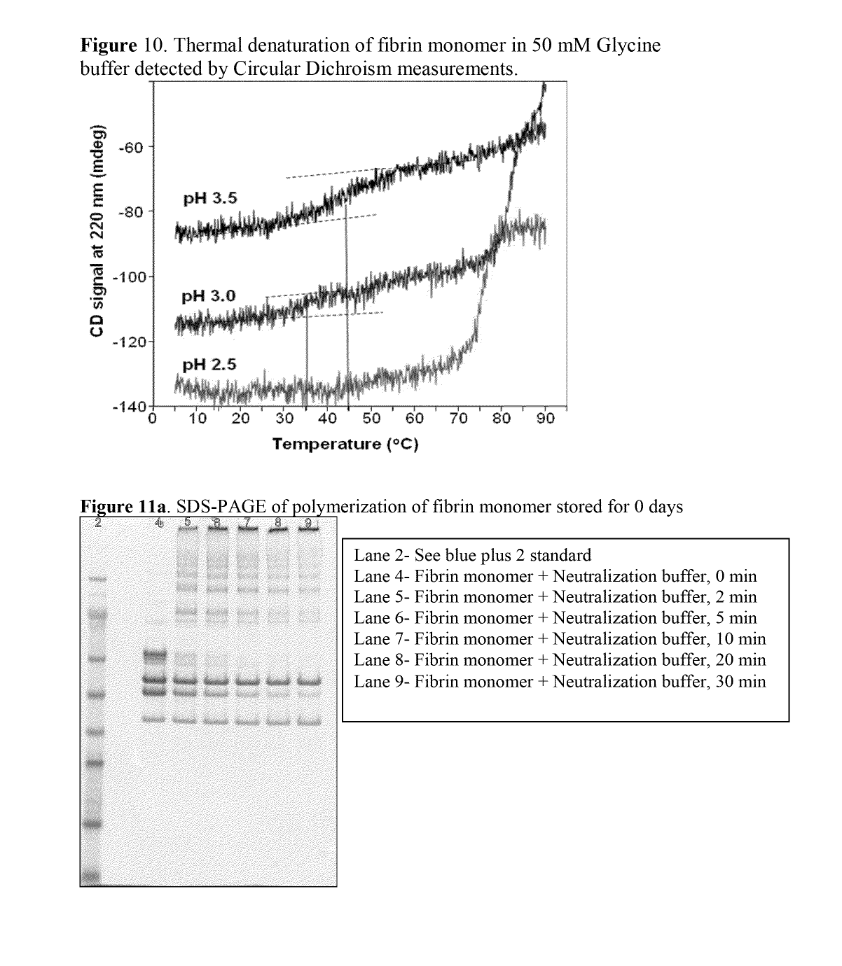

Fibrin monomer at 40 mg/mL in acetic acid solution at pH 3.1, 3.4, 3.8 and 4.2 was neutralized with a neutralization buffer resulting in a neutralized fibrin polymer at final pH 7. The clotting time for each monomer solution was 8 sec, 6 sec, 8 sec and 14 sec. respectively These experiments indicate that at the lower limit of pH value (3.1) and upper limit of pH value (4.2) at which fibrin monomer can be kept in solution clotting time is significantly delayed as compared to pH 3.4. Thus we concluded that 1) It is not feasible to maintain fibrin monomer in solution below pH 3.0 and over pH 4.2 since such monomer undergoes denaturation between pH 1.0 and 3.0 due to a very low structural stability and aggregates between pH 4.2 and 5.0 due to approaching isoelectric point, which for fibrin is at pH 5.6; 2) To maintain fibrin monomer in solution the most appropriate pH range in which fibrin monomer preserves its structural and functional properties is between pH 3.4 and 4.0. 3) Within this range the clotting time is dependent on pH, being pH 3.4 the optimal is relation with the polymerization rate (FIG. 10. Thermal denaturation of fibrin monomer)

### Example 6

Stability of 150 days in solution when stored at 4° C. with no additives Purpose: To establish shelf live of the agent by demonstrating that the fibrin component does not degrade under refrigerated conditions (2° to 8°) for a period C and under standard conditions (22° C.).

Methods: To determine the shelf life of ACstrength fibrin monomer solution three different studies were carried out namely; Clotting time, Clottability, and SDS-PAGE. Aliquots of fibrin monomer solution was kept at 2-8° C. for 150 days, and tested once a week starting from 0 day. Each study procedure is briefly described below:

Clotting time: To determine the clotting time of fibrin monomer solution, 250 μl of fibrin monomer was mixed with 750 ul of neutralization buffer. The clotting time was noted using the timer and recorded. Three experiments were performed and averaged out.

Clottability: The fibrin polymer formed during clotting time study was kept for approximately 2 hours for maturation, squeezed to get the possible liquid out and the clottability was measured. Three experiments were performed and averaged out. The optical density of the liquid was measured in a spectrophotometer and the % Clottability was calculated as follows:

Concentration of fibrin monomer before polymerization−Concentration of fibrin monomer in the liquid*100

### Concentration of Fibrin Monomer Before Polymerization

SDS-PAGE: The polymerization ability of fibrin monomer solution was determined by SDS-PAGE. To conduct the study, 22 μl of fibrin monomer solution was mixed with 22 μl of neutralization buffer and incubated for 0, 2, 5, 10, 20 & 30 min. At each time point, the polymerization reaction was stopped with 100 μl of 8M urea and the sample was diluted with 800 μl of 4M urea. The polymer was dissolved by heating. 20 μl of each sample was then mixed with 6.67 μl of 4× loading buffer and applied on the gel. The gel was run for 35-40 minutes at 200V, stained for 1 hour and de-stained until the sample becomes clearly visible. The gel was then scanned and saved.

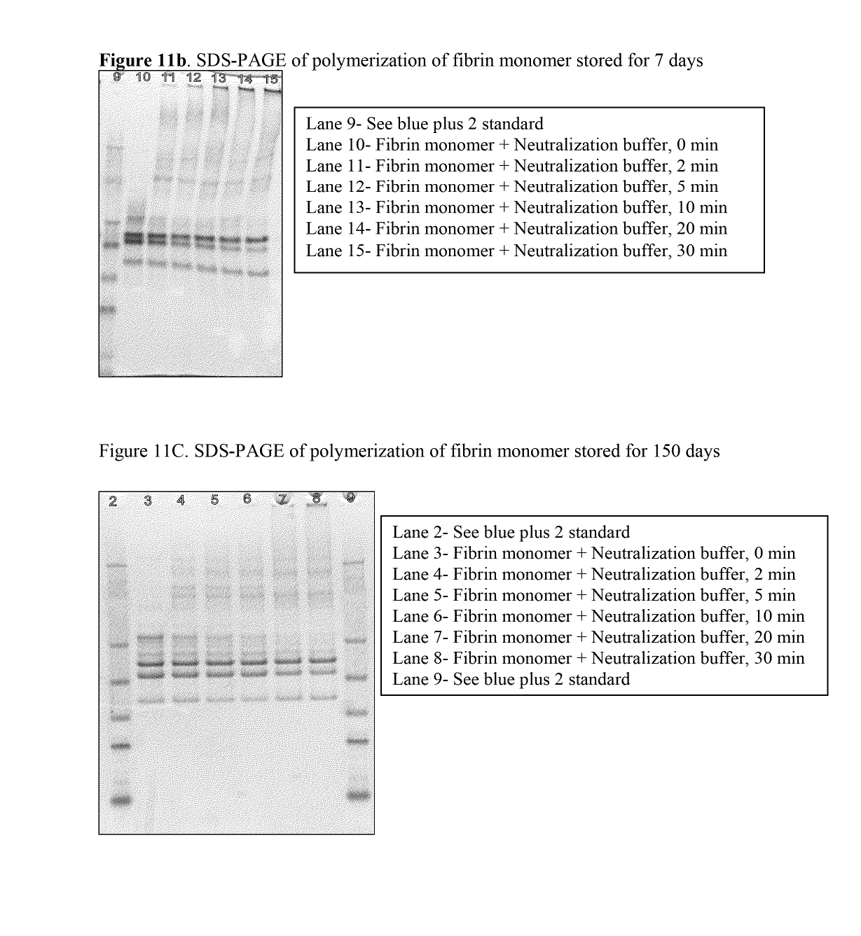

Result: The result of each study is shown below and in FIGS. 11a, 11b, and 11c.

### Clotting Time Result Chart

| Pre-clot | | Average | | | |
|---|---|---|---|---|---|
| concentration | Storage | clotting | Standard | | |
| of fibrin | condition | time | deviation | % CV | Study day |
| 12.38 mg/ml | 2-8° C. | 6 | 0 | 0 | 0 day |
| | | 6.33 | 0.58 | 9.1 | 15 day |
| | | 5.67 | 0.58 | 10.2 | 30 day |
| | | 6.67 | 0.58 | 8.7 | 45 day |
| | | 5.67 | 0.58 | 10.2 | 60 day |
| | | 9.67 | 0.58 | 6.0 | 75 day |
| | | 8.67 | 0.58 | 6.7 | 90 day |
| | | 8.33 | 0.58 | 6.9 | 105 day |
| | | 8.00 | 0.00 | 0.0 | 120 day |
| | | 10.00 | 0.00 | 0.0 | 135 day |
| | | 9.67 | 0.58 | 6.0 | 150 day |

### Clottability Result Chart

| Pre-clot | | | | | |
|---|---|---|---|---|---|
| concentration | | | | | |
| of fibrin | Storage | Average | | | |
| monomer | con- | Clottability | Standard | % | |
| solution | dition | (%) | deviation | CV | Study day |
| 12.38 mg/ml | 2-8° C. | 99.53 | 0.01 | 0 | 0 day |
| | | 99.52 | 0.01 | 0.0 | 15 day |
| | | 99.42 | 0.03 | 0.0 | 30 day |
| | | 99.50 | 0.03 | 0.0 | 45 day |
| | | 99.43 | 0.06 | 0.1 | 60 day |
| | | 99.42 | 0.02 | 0.0 | 75 day |
| | | 99.43 | 0.04 | 0.0 | 90 day |
| | | 99.40 | 0.03 | 0.0 | 105 day |
| | | 99.33 | 0.01 | 0.0 | 120 day |
| | | 99.26 | 0.02 | 0.0 | 135 day |
| | | 99.33 | 0.01 | 0.0 | 150 day |

### SDS-PAGE Result

SDS-PAGE analysis of monomer stored at 0, 7, and 150 days show no significant difference in alpha and gamma band and formation of d-dimmers and oligomers.

### CONCLUSION

Taking into account all data presented above it can be concluded that the ACstrength fibrin monomer solution is functionally stable upon storage at 2-8° C.

### Example 8

### Characterization of Fibrin Monomer Solution by HPLC

Purpose: To determine that ACstrength monomer II solution is monomeric, with no aggregates and degradation products by High Performance Liquid Chromatography (HPLC) analysis.

Materials: Fibrin monomers were prepared from two sources of fibrinogen, (GR) and CSL Behring (CSL) at pH 3.5. Final concentration of GR fibrin and CSL fibrin was 25 mg/mL. Bio-Rad Gel filtration standard was used as a reference standard.

Method: HPLC was performed on Agilent 1100 Series HPLC system. Namely, 50 L samples of GR and CSL fibrins were applied onto TSKgel G4000SW_{xL} column equilibrated with the same buffer. Flow rate was 1 mL/min and each fibrin sample was run in triplicates. Elution of fibrin was detected by measuring absorbance at 280 nm with reference at 360 nm. SEC filtration standards were used as protein standards, which were run in triplicates.

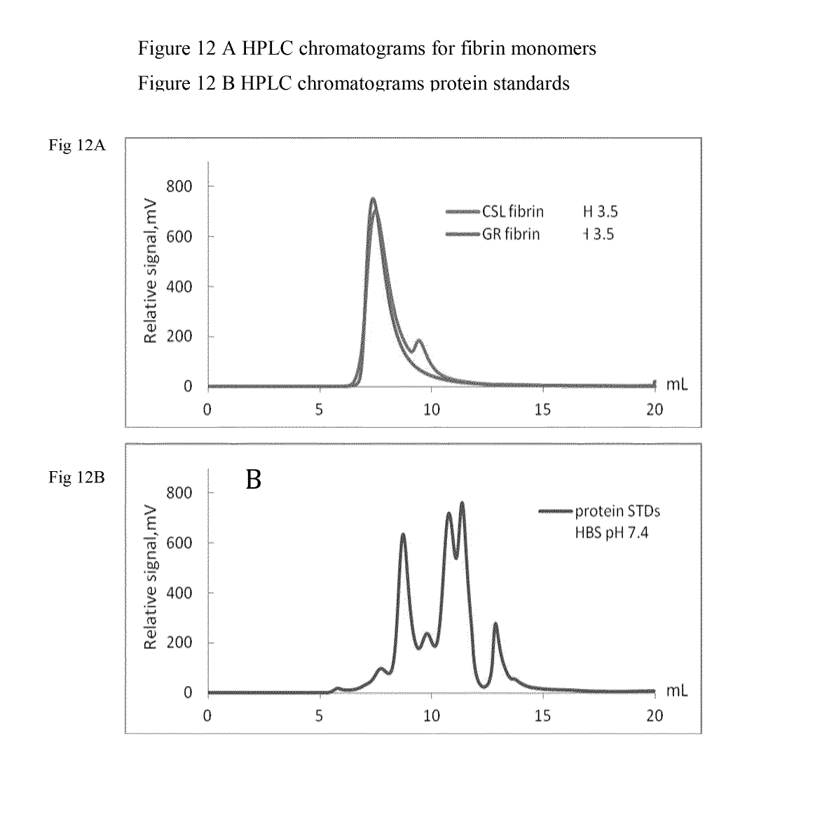

Results: HPLC chromatograms for fibrin monomers and protein standards are presented in FIG. 12 (a,b). Both, GR and CSL fibrins were eluted at the same retention time of 7.48 minutes and did not show presence of any aggregates. It should be noted that SEC profiles for both type of fibrins were consistent from batch to batch.

Conclusion: From HPLC size exclusion chromatography profile it can be concluded that the fibrin monomer is monomeric in nature and remains in solution at pH 3.5 with no degradation.

### Example 9

### SCANNING ELECTRON MICROSCOPY Characterization of the Fibrin Mesh Produced by Neutralization of ACstrength Monomer II Solution

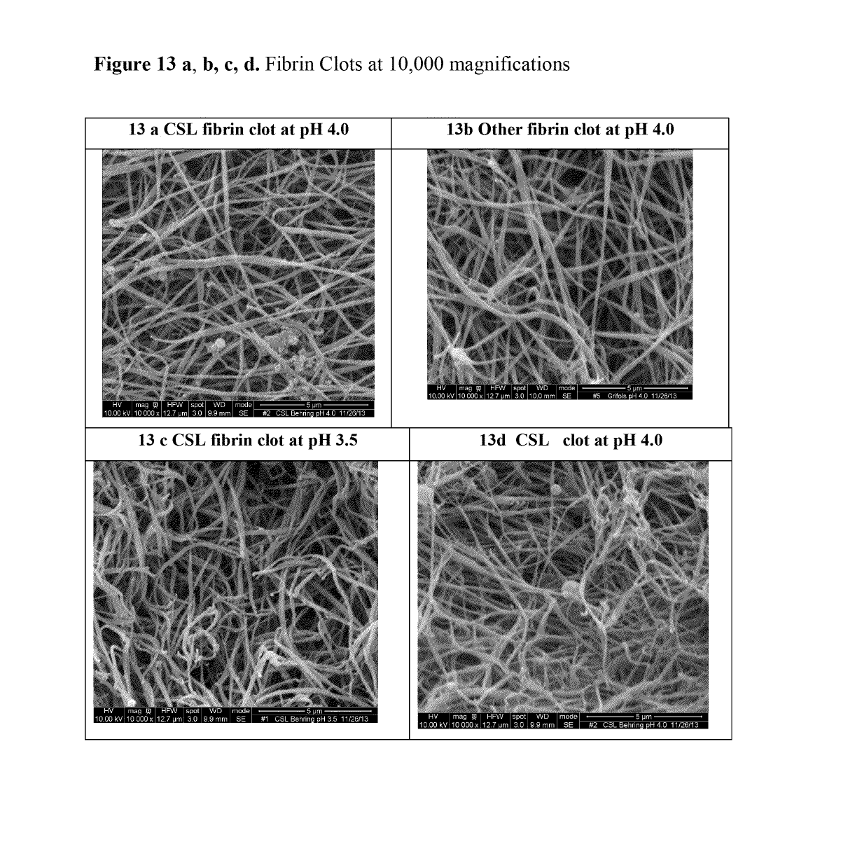

Clots prepared with GR or CSL fibrin were analyzed by scanning electron microscopy and the resulted SEM images at 10,000 magnifications are presented in FIGS. 13a, 13b, 13c, and 13 d. The images suggested similar network morphology in fibrin clot formed by different fibrinogen sources (GR and CSL) within a pH range of 3.5-4.0. Both clots revealed similarity in fiber length and in branch point density.

### CONCLUSIONS

The presented results clearly indicate that the characteristics of the polymer formed by neutralization of ACStrength Fibrin monomer II solution is independent of the source of fibrinogen, and that forms an opaque mesh with multiple branches characteristic of a strong clot. GR and CSL Behring fibrinogen form stable cross-linked clot of similar architecture (fibrin length, branch points density) under the same conditions.

### Example 10

### Polymerization Process with and without Calcium Independent Tranglutaminase Enzyme (Activa)

Purpose: To compare the gel strength (cross-linking) measured by rheometry and the effects on Fibrin monomer polymerization (dimmers and oligomers) by activated Factor XIII versus Ca Independent transglutaminase enzyme.

Materials and Methods: Fibrinogens from CSL Behring (CSL). Neutralization buffer (NB) for fibrin monomer at pH 3.5, Calcium independent transglutaminase enzyme (Activa), Factor XIII (FXIII) at 4700 U/mL from Enzyme Research Laboratories; Thrombin (Thr) at 500 NIH U/mL.

We conducted molecular chemistry assays to assess and compare the effects on Fibrin monomer polymerization and rheometry to compare the gel strength (cross-linking) by activated Factor XIII versus Ca Independent transglutaminase enzyme.

It is well established that FXIII in the presence of Ca²⁺ catalyzes fibrin monomer conversion into insoluble fibrin clot. In order to follow these reactions, fibrin monomer was subjected to calcium independent transglutaminase enzyme treatment and to activated factor XIII.

### Molecular Analysis

Activation of FXIII: 25.2 L of FXIII (120 U) were mixed with 20 L Thr (10 NIH U) and 254.8 L of HBS and incubated 10 min at 37° C. Final concentration of activated factor XIII (FXIIIa) was 400 U/mL.

Dilution of Fibrin-monomer samples for SDS-PAGE and Western Blot: 192 L of each fibrin sample was mixed with 108 L of the corresponding diluents to obtain concentration of 16 mg/mL.

Mixtures of Neutralization buffers with transglutaminase and FXIIIa:

| Sample | FXIIIa, L | Activa, L | NB, L |
|---|---|---|---|
| 1 | 32 | — | 240 |
| 2 | — | 80 | 192 |
| 3 | 32 | 80 | 160 |

Polymerization reaction: 25 L of diluted fibrin-monomer were mixed with 85 L of corresponding NB with transglutaminase and/or FXIIIa (samples 1, 2 or 3). Final concentration of fibrin in the mixture was 3.64 mg/mL, FXIIIa—36.4 U/mL, and Activa—68 mg/mL. Samples were incubated for 1, 5 and 10 min at 37° C., were thoroughly mixed with 0.1 mL of 8 M urea, 5% SDS, 5%—Mercaptoethanol and then diluted with 0.8 mL of 4M urea, 2.5% SDS, 2.5%—Mercaptoethanol. The final mixtures were incubated at 70° C. for 30 min.

Samples for SDS-PAGE analysis: 20 L of the samples were mixed with 7 L of NuPAGE sample buffer (4×). 17 L of each sample was loaded onto 12 well 4-12% SDS gel. Gels were run at 200V for 40 min then were rinsed with water (10 min) and stained with an Imperial Protein Stain (Thermo Scientific).

Samples for western Blot analysis: 10 L of the SDS-PAGE samples (left over) were further diluted with 40 L of NuPAGE sample buffer (1× diluted with H₂O). 10 L of each sample was loaded onto 12 well 4-12% SDS gel and ran at 200V for 40 min, transferred to the nitrocellulose membrane. iBlot Gel Transfer Stacks nitrocellulose (Invitrogen) were used, Program P3, 8 min. The membrane was blocked with 5% milk in TBS-T for 45 min, then was incubated with 1/5000 diluted anti fibrinogen antibodies-HRP (Enzyme Research Laboratories) overnight and developed with SuperSignal (Thermo Scientific).

Rheometry: To measure gel strength: G′ “rheometry” of fibrin clots was measured according to rheology protocol using Carri Med Rheometer, TA instruments, model CSL²₅₀₀. Fibrin monomer at 25 mg/mL was mixed with neutralization buffers in volume ration 1 to 1 to create fibrin clot containing 12.5 mg/mL of fibrin. In case of the clot with cross-linked fibrin monomer the final concentration of Activa was 5%.

### Results

Analysis of fibrin cross-linking by SDS-PAGE and Western blot. Assays compared a) fibrin and fibrinogen crosslinking by Factor XIII and calcium independent transglutaminase enzyme at 1, 5 and 10 Minutes.

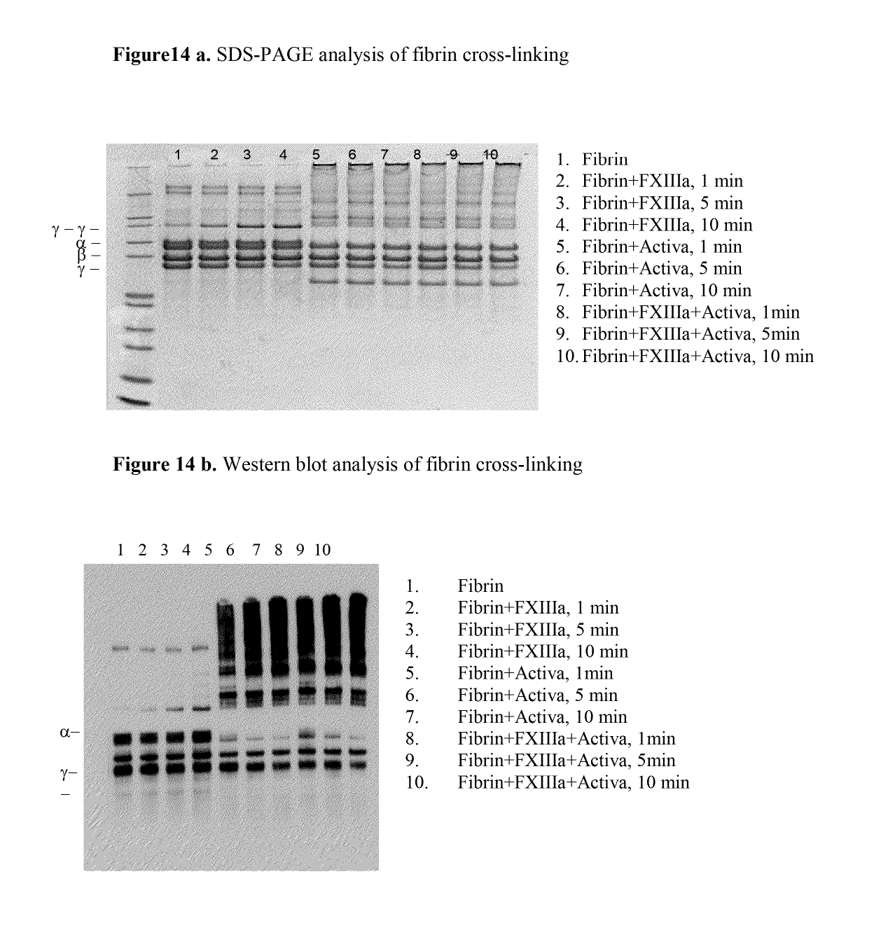

The cross-linking and stabilization of fibrin monomer solution at pH 3.5 by neutralization buffer containing Factor XIIIa and/or Ca Independent transglutaminase enzyme (Activa) analyzed by SDS-PAGE and Western blot are presented in FIGS. 14a and 14b respectively.

Neutralization of fibrin with buffer with calcium independent transglutaminase enzyme incorporated generated a number of cross-linked fibrin molecules (lanes 5 to 10) when compared with lanes 1 to 4 containing control sample of fibrin. Furthermore, fragmented derivative products (FDP) of lower molecular weight bands also participate in crosslinking, in addition to high molecular weight dimmers and tetramers.

The figures show the formation of strong gamma dimmers during fibrin cross-linking with calcium independent transglutaminase enzyme and factor XIII at 1 minute. At this time gamma dimmers are not yet present in the fibrinogen sample.

Rheometry measurements: The rheometry measurements of polymerized fibrin (fibrin clot) in the presence or absence of 5% of calcium independent transglutaminase enzyme (ACTIVA) produces a gel that is 300$% stronger than without ACTIVA (FIG. 14C).

### CONCLUSIONS

Rheometry data demonstrated that fibrin polymer cross-linked by Ca independent transglutaminase produced stronger clot in terms of rheometry values than in the absence of the enzyme. Also it was established the polymerization of AC Strength monomer II solution in the presence of Ca independent transglutaminase produces a crosslinked polymer showing formation high molecular weight dimmers and tretramers within one minute of reaction.

### Example 11

### Experiments in Animal Models

### 11. 1. We Conducted Studies on Debridement of Necrotic Tissue Following Severe Burns in the Swine (Pig) Model.

Study objectives: Compare ClotSpray versus epinephrin (standard of care) using to stop bleeding from cutaneous debrided necrotic tissue in the swine.

Method: Four female Yorkshire crossbred swine, age 2.5 months, weighing 37±2 kg, were used. The protocol was approved by the Institutional Animal Care and Use Committee of TMCI. Each animal were subject to three 10″ by 4″ debridement of burn injury in Pig skin (dorsal) with a dermatome. Pig skin has been shown to have similar histological and physiological properties to human skin and has been suggested as a good model for human skin permeability. After a 1 minute of injury allowing profuse bleeding, 8 ml of ClotSpray at a concentration of 40 mg/ml (Monomer Vs Buffer 1:3) was sprayed over the wounds in two of the experimental subjects. In the two control animals sponges pads soaked in 1:100,000 topical epinephrine solution was applied on the wounds to control hemostasis.

Results: hemostasis was achieved in all two wounds on experimental animals within 20 seconds (median of 22.2±2. sec). Hemostasis was achieved in the 2 control animals treated with epinephrine within 2.4 minutes. (median of 1.4±1. min)*

### 11.2 Evaluation of ClotSpray for the Control of Bleeding in Liver Biopsy

The purpose of this study is to determine if ClotSpray can stop light to moderate diffuse bleeding within 1 minute of application, and compare its efficacy to a standard fibrin sealant made of fibrinogen and thrombin, when used as an adjunct to hemostasis.

Methods: Four female Yorkshire crossbred swine, age 2.5 months, weighing 37±2 kg, were subjected to a liver biopsy with a 18-biopsy punch. A single suture of 3-0 absorbable material was paced to dose the wound. The animals were divided into 2 groups: 1) a 8 ml of ClotSpray at concentration of 40 mg/ml was aerosolized in the wound area; and 2) 8 ml of standard fibrin sealant made of fibrinogen concentration of 80 mg/ml and thrombin at concentration of 500 U/ml was sprayed over the wound. After therapy procedures were performed, lactated Ringer's solution resuscitation was then performed in both groups to maintain the mean arterial pressure at 70 mm Hg for 1 hour. All animals achieved hemostasis within 2 minutes of application. The ClotSpray group showed a lower time to hemostasis (20.4±2.2 sec versus 0.46±8.2 s) than the control group. Conclusions. ClotSpray significantly decreased time to hemostasis and consequently blood loss in a pig model of active liver bleeding. It provides a simple and quick method to control blood loss in liver injuries with active bleeding. ClotSpray significantly decreases the bleeding time at lower concentration of 40 mg/ml against 80 mg/ml for fibrin sealant. Furthermore the fact that ClotSpray does no contain thrombin allows for the self-recovery of blood lost and represents a significant advantage.

### 11.3 Ptygerium Surgery in Rabbit Eye for Glue.

Simulation of Pterygium excision by removal of conjunctival tissue approximately 4-6 mm from the limbus, and replacement with amniotic membrane was performed in both eyes of 3 rabbits. The Amniotic membrane was glue in place by using one single drop of ACstrength fibrin monomer II solution mixes with neutralization buffer in a proportion 1:1 through a dual syringe. The animals were survived for 72 hours in order to observe the efficacy of the attachment and detachment rate of the membrane.

Results: Animals were observed at 72 hs. All membranes remained attached to the conjunctiva.

### 12. Safety Studies

Four swine, Group 1 who underwent a liver biopsy liver via open laparotomy and were treated with 8 ml of clotspray (4.3) were euthanized at week four after completion of surgery. Similarly, two controls treated with fibrin sealant were followed and euthanized after 4 weeks, and necropsy was performed and tissue samples from main organs were obtained. Organs were collected, fixed in 10% formalin and embedded in paraffin blocks. Histologic sections were stained with Hematoxylin and Eosin and examined at 100× and 400× in optical and microscopes. These slides were evaluated by a Board-certified veterinary pathologist.

The safety of the agent was assessed through the evaluation of toxicity, physiological adverse effects, biocompatibility, delayed hematoma and/or edema formation and immunological risks. Physiological and pathological observations included: Mortality/morbidity; Body weight, Food consumption, Organ weights:

12.1 Acute Toxicity was assessed by blood chemistry, macroscopic evaluation at necropsy and by histological studies.

12.2 Irritation of tissues and tissue vessels to which the agents ere in contact was assessed looking for evidence of acute and/or chronic inflammation as signs or irritation in the histology.

12.3 Thrombosis, fistula, and abscess formation was assessed for all organs

12.4 Assessment of delayed hematoma: Risk of subcapsular or parenchymal hematoma formation. Delayed hematoma and edema formation was observed macroscopically and histologically at 21 days after application. Small hematoma formation is defined as a visible or palpable mass of ≧4 cm in diameter without associated sequalae.

### Histology

We analyzed the histological damage in lungs, kidneys, liver, spleen from all treated after 4 weeks of surgery, and compared sample treated with ClotSpray to control (treated with standard fibrin sealant) Data on inflammation included apoptosis and leukocyte infiltration. Inflammation and edema formation was also assessed histologically.

Evaluation of Immunologic Responses Potential antibody responses to ClotSpray were evaluated.

Methods: Serum samples were collected from experimental animals subjected to liver biopsy pre- and post-treatment on Day 0, and Day 31 days post-surgery and stored frozen at −20° C. until analysis. Antibodies generated to the components that are used in the formulation of ClotSpray, namely Fibrin, and Activa ClotSpray were tested by enzyme-linked immunosorbent assay (ELISA).

To detect antibodies that might be produced in swine against components of ClotSpray, a sandwich ELISA (enzyme linked immunosorbent assay) was constructed. The bottom surfaces of 96-well microtiter plates were coated overnight at 4° C. with Fibrin (10 mg/ml in PBS, pH 7.0, Sigma-Aldrich) or Activa (n 10 mg/ml in PBS, pH 7.0, Sigma-Aldrich). All wells were washed 5 times with PBS. Samples of swine serum were applied at 1:20 final dilution in PBS, incubated for 1 hr at room temperature and washed 5 times with PBS. Enzyme (horseradish peroxidase)—conjugated rabbit antibodies to pig IgG (Sigma-Aldrich) were applied to all wells at 1:20 dilution in PBS, incubated for 1 hr at room temperature, and washed 5 times with PBS. Substrate was prepared by dissolving one capsule of substrate (2,2′-Azino-bis(3-ethylbenzothiazoline-6-sulfonic acid)diammonium salt, 10 mg/capsule, Sigma-Aldrich) in 100 ml of phosphate-citrate buffer, pH 5.0, and adding H₂O₂ (0.25 ml of 3% solution). Following incubation for 10 min at room temperature, optical density at 405 nm was determined using a BioTek EX800 microplate reader.

Each targeted component was diluted 1:100 in phosphate-buffered saline (PBS), pH 7.4, coated onto microtiter plate wells, and incubated overnight at 4° C. The wells were blocked with 0.25% (wt/vol) nonfat dry milk/0.2% Tween 20 in PBS (blocking buffer) and then incubated with 50 μL of a 1:10 dilution of animal serum in blocking buffer for 1 hour at 37° C. Bound IgG and IgM were detected as standard ELISA system for secondary antibody.

The normal range was determined with 5 normal animal sera. An elevated antibody level is defined as greater than two standard deviations above the normal mean. Each plate included wells incubated with all reagents except for the diluted serum, which provided the background absorbance that was subtracted from all results.

### Results:

In this study adhesions in the abdominal wall were expected and found to be within the expected range for these types of injury and treatment. Adhesions were of similar grade than controls treated with Evicel. Other adhesions identified in other organ sites during this studies were found to be unassociated with the test article, as they were far removed from the injury site.

In the majority of pigs, there was no evidence of edema, thromboembolism, or locate irritation of any surrounding organ.

Summary reports of blood chemistries for each animal indicate that the values were within normal limits with only a few minor changes. Despite liver in jury, the liver function and renal function tests were within normal limits.

In regards to the evaluation of antibody immune response, there were no significant differences in OD readings observed with sera collected on day 0, or day 31 from control Antibodies against fibrin were at or below detection at 31 days, Antibodies against Activa were detected at low levels at day 31, demonstrating that the antibody responses are not intense or of long duration. Overall, the studies of antibody responses to clot foam indicate that the immunogenic components of ClotSpray, fibrin and Activa elicit variable, but generally low titer antibodies that are not expected to persist over long periods of time post treatment or cause any serious deleterious effects.

### Necropsy:

| Pig | | General | | Other | Inflammatory |
|---|---|---|---|---|---|
| Number | Treatment | Condition | Adhesions | organs | response |
| #05372 | 8 ml ClotSpray | Normal | grade 2-3 adhesion | normal | |
| (28 days) | Liver Biopsy | | from abdominal | | |
| | | | wall to organs | | |
| #05373 | 8 ml ClotSpray | normal | Grade 3 adhesions | normal | No |
| (28 days) | Liver Biopsy | | from abdominal | | hematoma |
| | | | wall to liver and | | |
| | | | spleen | | |
| #05374 | 8 ml ClotSpray | normal | grade 2-3 adhesion | normal | No |
| (28 days) | Liver Biopsy | | from abdominal | | hematoma |
| | | | wall to organs | | |
| #05375 | 48 ml ClotSpray | normal | Moderate | normal | No |
| (28 days) | Liver Biopsy | | adhesions to | | hematoma |
| | | | abdominal wall | | |

Conclusion: Both the control (treated with Evicel) and treated animals with ClotSpray contained mild chronic inflammation and fibrosis at the site of the abdominal wall injury. No adverse events related to the test agent were observed. Experimental pigs produced no detectable antibodies against ClotSpray. We conclude that the testing agent is non-toxic, non-immunogenic, and safe for use in humans.

13 Pharmacokinetic profile of the agent through Biodegradation studies. Elimination through biodegradation by proteolytic enzymes was determined in vivo.

Method: To examine the fate of ClotSpray in vivo, the experimental batch used in liver biopsy was prepared with a fluorescein-tagged human fibrinogen as tracer. This preparation was applied to the four animals tested in experiment described in section 11.2 which were euthanized at 4 weeks following application. Once animals were sacrificed, organs were collected, fixed in 10% formalin and embedded in paraffin blocks. Histologic sections were examined at 100× and 400× in fluorescence microscope. The elimination of ClotSpray was determined by the level of fluorescence in tissues obtained from the biopsy area in the liver as compared with tissues in from another liver lobe, observed at 4 weeks.

### Results:

Microscopic examination of tissue samples from Day 31 demonstrated that ClotSpray treatment, the remaining fluorescence was at or near background levels among ClotSpray samples,

Normal tissue appearance was observed in most tissues of control and ClotSpray treated animals Microscopic examination under UV light showed that remaining trace fluorescence was trapped in interstitial spaces liver on day 31, however hepatocytes were free of any fluorescence and had normal appearance.

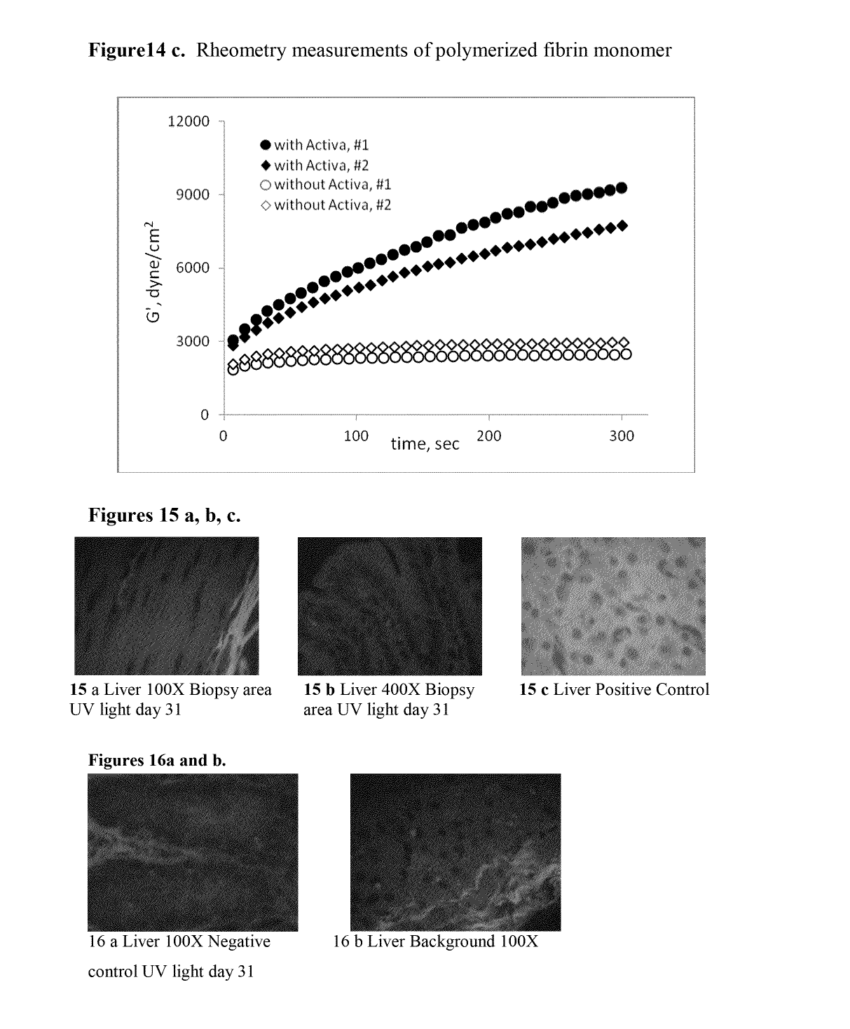

Histological sections of the tissues from ClotSpray treated animals and controls, taken at 4 weeks after treatment, are presented in FIGS. 15 and 16.

Conclusion ClotSpray was eliminated in all organs within 4 weeks of application

### Example 14

### Sterile Preparations of ClotSpray were Studied

Purpose: To assess and validate the method for sterilization of ClotSpray,

Method: The acidic Fibrin Monomer was sterile filtered in a biological safety cabinet using a Nalg-Nunc 500 mL device (Cat #450-0045, nitrocellulose membrane, 0.45 m filter).

The general experimental protocol included preparation of sample solutions which were then plated on Potato dextrose agar (PDA, Sigma-Aldrich, Cat#P2182) and Tryptic soy agar (TSA, Sigma-Aldrich, Cat#T4536) gels in Petri dishes for growth. The PDA and TSA gels were incubated and observed at the indicated periods of time for colony growth (mold and/or bacteria).

The sample was incubated for 30 min at 37° C. and evaluated for colony growth using the naked eye at the time periods indicated in the Results and Discussion section. The samples were run in duplicate or triplicate with multiple samples indicated with a 1, 2 and 3 designation in data tables. The scale used for evaluation is as follows:

TABLE 2
Colony Count Key

| Symbol | Count |
|---|---|
| − | No visible growth |
| + | 1-199 visible colonies |
| ++ | 200-399 visible colonies |
| +++ | >400 visible colonies |

### Results

Table 3 shows the results of studies of microorganism growth analysis on PDA and TSA of the sterile components of FIBRIN_ClotFoam.

TABLE 3
Sterilization Studies by Bacterial Growth on PDA/TSA at 37° C. — —

| | | Potato Dextrose Agar (PDA) | | | | | | | | Tryptic Soy Agar (TSA) | | | | | | | |
|---|---|---|---|---|---|---|---|---|---|---|---|---|---|---|---|---|---|
| Time Elapsed (days) | 23 | 1 | 2 | 3 | 4 | 5 | 6 | 7 | 11 | 1 | 2 | 3 | 4 | 5 | 6 | 7 | 11 |
| Sample | #s^{$} | | | | | | | | | | | | | | | | |
| C** | 1 | | | | — | | | — | — | | | | — | | | — | — |

The growth data indicate that sterile components yielded no significant growth even after 11 days.

### Example 15

### Biocompatibility

Two ClotSpray preparations were made under sterile conditions-were tested. These preparations were tested for biocompatibility with human fibroblasts (HF) and human epithelial cells (A549 cell line, ATCC).

Normal human fibroblasts (HFs) were obtained from a commercial source and cultures established in 60 mm tissue culture plates in Dulbecco's modified Eagle's medium supplemented with 10% fetal bovine serum and maintained at 37° C. in a humidified 5% CO₂ atmosphere (CO₂ incubator). Human epithelial cell line A549 was maintained in Minimal Essential Medium supplemented with 10% fetal bovine serum and 2 mM glutamine. When fibroblast and epithelial cell cultures reached subconfluence, control and sodium benzoate Clotcake preparations were sumerged into individual dishes. The cultures were returned to the CO₂ incubator and examined daily for a total of five days. ClotSpray material and medium was removed from all cultures, and adherent cells were stained with crystal violet (0.1% in 2% ethanol).

### Results

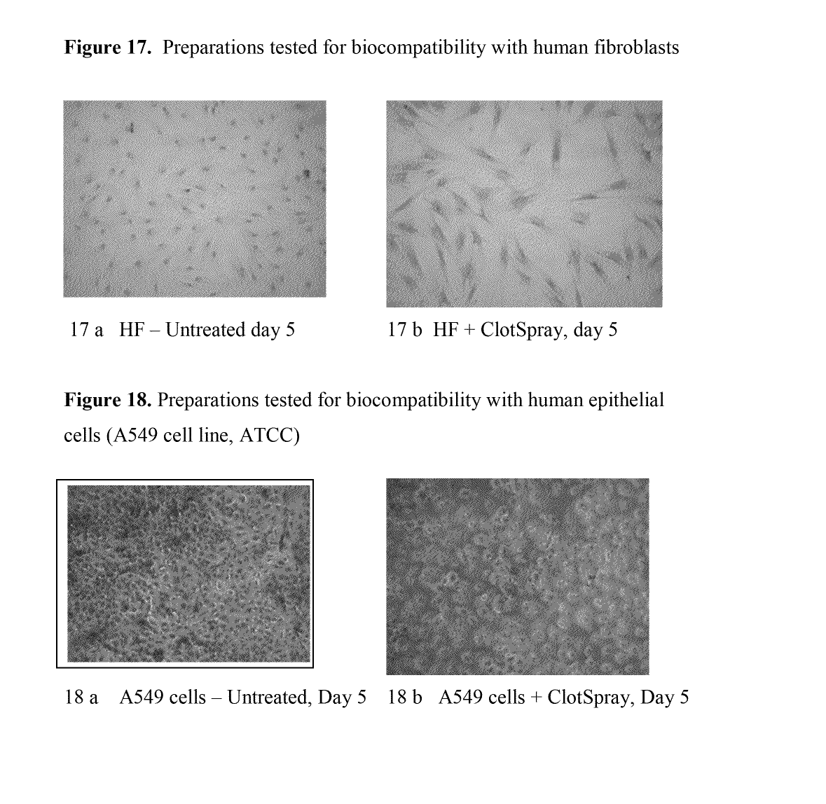

The main observation was a total absence of damage or toxicity to the cells, and absence of any bacterial or fungal contamination. In human fibroblast cultures exposed to ClotSpray preparations, the cells appeared slightly larger or more spread out than in control untreated cultures. FIGS. 17 (a, b) and 18 (a,b).

Conclusion: ClotSpray is biocompatible, and does not affect, but rather stimulate, the growth and differentiation of cells; which is an important attribute in wound healing agents.

### LITERATURE CITED

1. Mosher D F, Schad P E. Cross-linking of fibronectin to collagen by blood coagulation Factor XIIIa. J Clin Invest. 64:781-7. 1979.
2. Rousou J, Levitsky S, Gonzalez-Lavin L, Cosgrove D, Magilligan D, Weldon C, Hiebert C, Hess P, Joyce L, Bergsland J, et al. Randomized clinical trial of fibrin sealant in patients undergoing resternotomy or reoperation after cardiac operations. A multicenter study. J Thorac Cardiovasc Surg. 97:194-203. 1989
3. Dunn C J, Goa K L. Fibrin sealant: a review of its use in surgery and endoscopy. Drugs. 58:863-86. 1999
4. Schelling G, Block T, Gokel M, Blanke E, Hammer C, Brendel W. Application of a fibrinogen-thrombin-collagen-based hemostatic agent in experimental injuries of liver and spleen. J Trauma. 28:472-5. 1988.
5. Paul A Janmey, Jessamine P Winer and John W Weisel. Fibrin gels and their clinical and bioengineering applications J. R. Soc, interface 6 Jan. 2009 vol, 6 no. 30 1-10.
6. (Fang N, Zhu A, Chan-Park M B, Chan V. Adhesion contact dynamics of fibroblasts on biomacromolecular surfaces. Macromol Biosci. 2005 Oct. 20; 5(10):1022-31.)
7 Action of fibrinogen degradation products and fibrin monomer soluble complexes on platelet aggregation. Larrieu M J. Scand J Haematol Suppl. 1971; 13:273-9.
8. Francis C W, Marder V J. Increased resistance to plasmic degradation of fibrin with highly crosslinked alpha-polymer chains formed at high factor XIII concentrations. Blood. 1988 May; 71(5):1361-5
9. Okumura N, Terasawa F, Haneishi A, Fujihara N, Hirota-Kawadobora M, Yamauchi K, Ota H, Lord S T. B:b interactions are essential for polymerization of variant fibrinogens with impaired holes ‘a’. J Thromb Haemost. 2007 December; 5(12):2352-9. Epub 2007 Oct. 8
10. Michael R. Falvo, Oleg V. Gorkun, and Susan T. Lord The molecular origins of the mechanical properties of fibrin: Biophys Chem. November 2010; 152(1-3): 15-20.
11. Belitser V A, Varetskaja T V, Malneva G V. Fibrinogen-fibrin interaction. Biochim Biophys Acta. 1968 Feb. 19; 154(2):367-75
12. Biophys J. 2010 May 19; 98(10): 2281-2289. doi: 10.1016/j.bpj.2010.01.040 Structural Hierarchy Governs Fibrin Gel Mechanics Izabela K. Piechocka, Rommel G. Bacabac, Max Potters Fred C. MacKintosh, ‡ and Gijsle H. Koenderink

What is claimed is:

1. A method for producing an aerosolized hemostatic sealant comprising 3 components described in portions (a-c), for cessation of blood loss, gluing, or sealing injured tissue of a patient's body
a) a first component in a liquid form comprising of fibrin monomer in an acidic solution that polymerizes upon change of pH from acidic to neutral;
b) a second component in liquid form comprising a neutralization HEPES buffer with calcium chloride; and
c) a third component in powder form comprising calcium independent tranglutaminase enzyme in the form of ACTIVA.

2. A method of claim 1 comprising the steps of:
i. producing a fibrin monomer in acetic acid solution
ii. producing a neutralization buffer
iii. applying a layer of a biodegradable polymer film through an aerosol-like device that mixes the said components, or alternatively by a dual barrel syringe.

3. The method set forth in claim 2, wherein the fibrin monomer solution thereof called AC Strength Fibrin II monomer, is produced by dialyzing fibrin II polymer against acetic acid to obtain a monomeric solution at a concentration of 36 mg/ml to 40 mg/ml of fibrin in acetic acid.

4. The method set forth in claim 2, wherein the fibrin II polymer is made by first dialyzing fibrinogen at the concentration of 40-50 mg/ml against 20 mM glycine buffer, pH 8.5, and 450 mM sodium chloride, and subsequently cleaving the fibrinogen solution with thrombin at a concentration of 100 NIH U/ml at a ratio of 50 ml fibrinogen:1 ml thrombin.

5. The method set forth in claim 3, wherein the concentration of fibrin monomer in solution can be reduced to 20 mg/ml by dilution in acetic acid or increased to 60 mg/ml by ultrafiltration in AMICON tubes.

6. The method set forth in claim 3, wherein the fibrin monomer in solution is 99.8% monomeric with no aggregates or degradation products.

7. The method set forth in claim 2, wherein the fibrin monomer is dynamically or counter-current dialyzed to increase by 300% the rate of fibrin dissolution into acetic acid.

8. The method set forth in claim 7 where dialysis is made through a 100 kDa membrane that depletes the AC Strength monomer solution of thrombin and plasminogen.

9. A method set forth in claim 2, wherein the pH of fibrin monomer solution is in the range of 3.4 to 3.8, ready for polymerization upon an abrupt increase in pH to about 7.0.

10. A method set forth in claim 2, wherein the neutralization buffer when mixed to AC Strength Fibrin II monomer solution in the proportion of 1:1 to 1:3 induces an abrupt increase in pH to about 7.0 inducing a rapid polymerization.

11. A method set forth in claim 10 wherein the neutralization of AC Strength Fibrin II monomer solution inducing the polymerization occurs within 5 seconds.

12. A method set forth in claim 10 wherein the neutralization of AC Strength Fibrin II monomer solution by the neutralizing buffer containing at least 2 grs of tranglutaminase enzyme in the form of ACTIVA which covalently crosslinks at least two of the three chains of the resulting polymer.

13. A method set forth in claim 12 wherein the tranglutaminase enzyme in the form of ACTIVA can be placed, in solid form, in the downstream fluid path of the liquid components, during the application.

14. A method set forth in claim 10 wherein the neutralization of AC Strength Fibrin II monomer solution by the neutralizing buffer and ACTIVA produces a crosslinked polymer of opaque fibrin fibers with a gel strength measured as sheer stress greater than 15,000 dyn/cm²

15. A method set forth in claim 10 wherein the neutralization of AC Strength Fibrin II monomer solution by the neutralizing buffer with ACTIVA over fresh blood produces a blood clot with a gel strength measured as sheer stress greater than 35,000 dyn/cm² or twice the strength of fibrin sealant produced by cleavage of fibrinogen by thrombin.

16. A method set forth in claim 2 by which the solutions can be stored at 2° C. to 8° C. for at least 150 days.

17. A method of claim 1 wherein the polymer produced by neutralization of AC Strength Fibrin II monomer solution can be dispensed to a surgical site by an aerosol-like device or a dual syringe displacement system to provide hemostasis or to bind and seal tissue.

18. The method set forth in claim 17, wherein the aerosol-like device is an applicator that uses aerosol can and modified aerosol valve technology to displace a piston within a product container or luer-lock syringe containing the biomaterial component, as to avoid contact between the biomaterial and the gas.

19. The method set forth in claim 17, where the product container is sealed by a floating piston and placed inside a pressurized vessel such as an aerosol.

20. The method set forth in claim 17, wherein the product container is connected to the aerosol valve, resulting in displacement of the piston within the product container when the container is exposed to atmospheric pressure via actuation of the aerosol valve, referred to here after as the aerosol assembly, resulting in ejection of the biomaterial or multiple biomaterials concurrently into a common manifold.

21. The method set forth in claim 20 wherein the manifold receives the biomaterial products from the aerosol assemblies for mixing and spraying them.

22. The method set forth in claim 20, wherein the manifold can be used to simultaneously actuate the displacement of biomaterial from each aerosol assembly attached to the manifold.

23. The method set forth in claim 20, wherein the pressure can be individually established for each individual aerosol assembly in order to obtain specific flow rates of biomaterial into the manifold from each aerosol assembly, resulting in different final ratios of the biomaterial mixture.

24. The method set forth in claim 18, wherein the product container inside the aerosol assembly could be a biocompatible compressible bag not requiring a piston for biomaterial displacement but where in displacement is achieved by the surrounding pressure inside the aerosol can compressing the bag when the interior pressure of the compressible bag is exposed to atmospheric pressure, upon actuation of the aerosol valve, resulting in ejection of the biomaterial into the common manifold for subsequent mixture with other biomaterial components.

25. The method set forth in claim 17, wherein the surgical site includes at least one of the patient's skin, abdominal cavity, thorax, cardiovascular system, lymphatic system, pulmonary system, ear(s), nose, throat, eye(s), liver, spleen, cranial, spinal, maxillo-facial, bone, tendon, pancreas, genito-urinary tract or alimentary tract.

26. The method of claim 1 wherein said composition produces a functional blood clot (prevents rebleeding) within 1 to 5 minutes from the moment that it is applied to the wound.

27. A method set forth in claim 1 wherein the hemostatic sealant is biocompatible or not interfering with cell growth, biodegradable within 60 days, non-toxic, non-immunogenic and safe for use in humans.

28. A composition of claim 1 wherein the acetic acid in the AC Strength Fibrin monomer II solution has a pH of 3.4 to 3.8.

29. A composition of claim 1 wherein the neutralization buffer comprises ingredients described in portions (a-d) for neutralizing AC Strength Fibrin II monomer kept in acetic acid solution to pH 7.0 in a volume proportion of 1:1
a) 100 mM HEPES
b) 300 mM NaCl
c) 20 mM CaCl₂
d) pH of this buffer is adjusted to 7.5

30. A composition of claim 1 wherein the neutralization buffer comprises ingredients described in portions (a-d) for neutralizing AC Strength Fibrin II monomer to kept in acetic acid solution to pH 7.0 in a volume proportion of 1:3
a) 300 mM HEPES
b) 1200 mM NaCl
c) 40 mM CaCl2
pH of this buffer is adjusted to 8.2

31. A composition of claim 1 wherein the mixture contains at least 2 grs of tranglutaminase enzyme in the form ACTIVA per 10 ml of solution.

32. The composition of claim 1 wherein their application over tissue is biocompatible and not toxic and does not elicit necrotic damage.

33. The composition of claim 1 wherein the application does no elicit antibody response.
